(19) United States
(12) Patent Application Publication
(10) Pub. No.: US 2010/0255550 A1
(43) Pub. Date: Oct. 7, 2010

(54) Regulatory Factors Controlling Oil Biosynthesis In Microalgae And Their Use

(75) Inventors: CHRISTOPH BENNING, EAST LANSING, MI (US); RACHEL MILLER, HOLT, MI (US); ERIC R. MOELLERING, LANSING, MI (US)

Correspondence Address: MEDLEN & CARROLL, LLP, 101 HOWARD STREET, SUITE 350, SAN FRANCISCO, CA, 94105, US

(73) Assignee: BOARD OF TRUSTEES OF MICHIGAN STATE UNIVERSITY

(21) Appl. No.: 12/559,974

(22) Filed: Sep. 15, 2009

Related U.S. Application Data
(60) Provisional application No. 61/096,971, filed on Sep. 15, 2008.

(51) Int. Cl. C12P 7/64; C07H 21/04; C07K 14/00; C12N 15/74; C12N 5/10; C12N 1/13

(52) U.S. Cl. 435/134; 536/23.1; 530/350; 435/471; 435/419; 435/257.2

(57) ABSTRACT

The production of transgenic plant oils is described by transfecting plant host cells with heterologous transcription inducers. Such inducers are specific for endogenous biosynthetic plant oil genes, such that the inducers induce an overexpression of the plant oil genes. The overexpressed gene product (i.e., for example, diacylglycerol transferase) thereby results in a increased intracellular production of a plant oil (i.e., for example, triacylglycerol). In one embodiment, the transfected plant host cell can be a plant algae species (i.e., for example, Chlamydomonas reinhardtii).

### FIELD OF THE INVENTION

The present invention is related to biosynthetic oil compositions and methods of making thereof. The present invention contemplates using plant material (i.e., for example, algae) comprising recombinant transcription inducing factors for biosynthetic oil genes. In some embodiments, the inducing factors are transcription factor regulatory proteins.

### BACKGROUND

Plants have long been a commercially valuable source of oil. Traditionally, plant oils were used for nutritional purposes. Recently, however, attention has focused on plant oils as sources of industrial oils, for example as replacements for, or improvements on, mineral oils. Given that oil seeds of commercially useful crops such as Brassica napus contain a variety of lipids (Hildish & Williams, “Chemical Composition of Natural Lipids”, Chapman Hall, London, 1964), it is desirable to tailor the lipid composition to better suit other needs, for example use in recombinant DNA technology (Knauf, TIBtech, February 1987, 40-47).

The production of commercially desirable specific oils in plants on a large scale is limited in two ways. Some plant species make oils with very high levels of essentially pure, specific fatty acids, but these species are unable to be grown in sufficient quantities and of sufficient yield to provide a commercially valuable product. Other plant species produce sufficient amounts of oil, but the oil has low levels of the specific desired fatty acids. Nevertheless, the field of oil modification in plants is wide and a number of different products have already been designed. Rape oil containing lauric acid has been marketed, and soybeans with modified levels of unsaturated fatty acids are available. In some cases, the production of speciality oils seems to be straight-forward. In others, however, a number of unexpected complications have arisen which have hampered the production of plants capable of making some specific oils. For example, mutations in plant lipid synthesis genes are generally difficult to detect due to the pleiotrophic effects of mutations on plant hardiness and yield. Even if detected, proteins involved in pathways of interest have proved difficult to isolate due to their biochemical instability.

Where regulation of such proteins has been successfully altered, results generally do not coincide with expectations, presumably due to the effect of multiple converging pathways. Examples of such problems relating to the production of Arabidopsis producing petroselinic acid are disclosed in Ohlrogge, 13^{th} International Symposium on Plant Lipids, Seville, Spain: 219 & 801, (1998). Thus, there are considerable problems to be solved in achieving reliable, large-scale production of a range of commercially desirable oils. For example, what is needed is a high-throughput method to improve biosynthetic plant oils production by recombinant engineering of biosynthetic oil gene regulation.

### SUMMARY OF THE INVENTION

The present invention is related to biosynthetic oil compositions and methods of making thereof. The present invention contemplates using plant material (i.e., for example, algae) comprising recombinant transcription inducing factors for biosynthetic oil genes. In some embodiments, the inducing factors are transcription factor regulatory proteins.

In one embodiment, the present invention contemplates a composition comprising a nucleic acid sequence encoding a biosynthetic oil gene transcription regulator. In one embodiment, the nucleic acid sequence comprises SEQ ID NO:2 (TF1). In one embodiment, the nucleic acid sequence comprises SEQ ID NO:5 (TF2). In one embodiment, the nucleic acid sequence comprises SEQ ID NO:8 (TF3). In one embodiment, the nucleic acid sequence comprises SEQ ID NO:11 (TF4). In one embodiment, the nucleic acid sequence comprises SEQ ID NO:14 (TF5). In one embodiment, the nucleic acid sequence is derived from a plant algae. In one embodiment, the algae comprises Chlamydomonas reinhardtii.

In one embodiment, the present contemplates a composition comprising an amino acid sequence comprising a biosynthetic oil gene transcription regulator. In one embodiment, the amino acid sequence comprises SEQ ID NO:3 (TF1). In one embodiment, the amino acid sequence comprises SEQ ID NO:6 (TF2). In one embodiment, the amino acid sequence comprises SEQ ID NO:9 (TF3). In one embodiment, the amino acid sequence comprises SEQ ID NO:12 (TF4). In one embodiment, the amino acid sequence comprises SEQ ID NO:15 (TF5). In one embodiment, the amino acid sequence is derived from a plant algae. In one embodiment, the algae comprises Chlamydomonas reinhardtii.

In one embodiment, the present invention contemplates a method comprising: a) providing; i) a nucleic acid sequence comprising a biosynthetic oil gene transcription regulator; ii) at least one plant cell comprising a biosynthetic oil gene; and iii) a vector comprising a promoter capable of expressing the transcription regulator; b) ligating the nucleic acid sequence to the vector, wherein the nucleic acid sequence is operably linked to the vector; and c) transfecting the algae cell with the operably linked vector such that the nucleic acid sequence expresses the transcription regulator. In one embodiment, the method further comprises step (d) inducing the biosynthetic oil gene with the transcription regulator, wherein the biosynthetic oil gene expression is upregulated. In one embodiment, the gene expression is ectopic. In one embodiment, the biosynthetic oil gene is upregulated between 1.5-3 fold. In one embodiment, the biosynthetic oil gene is upregulated between 3.5-5 fold. In one embodiment, the biosynthetic oil gene is upregulated between 5.5-7 fold. In one embodiment, the biosynthetic oil gene is upregulated between 7.5-10 fold. In one embodiment, the biosynthetic oil gene encodes a diacylglycerol acyltransferase enzyme. In one embodiment, the enzyme produces a fatty acid. In one embodiment, the fatty acid comprises triacylglycerol. In one embodiment, the method further comprises step (e) collecting the fatty acid, thereby forming a biosynthetic oil. In one embodiment, the plant cell comprises an algae cell. In one embodiment, the algae cell comprises a Chlamydomonas reinhardtii cell.

In one embodiment, the present invention contemplates a transgenic plant cell line, wherein the line is established by tissue culture propagation. In one embodiment, the transgenic plant cell line comprises a nucleic acid sequence encoding a heterologous DGAT transcription regulator. In one embodiment, the nucleic acid sequence comprises SEQ ID NO:2 (TF1). In one embodiment, the nucleic acid sequence comprises SEQ ID NO:5 (TF2). In one embodiment, the nucleic acid sequence comprises SEQ ID NO:8 (TF3). In one embodiment, the nucleic acid sequence comprises SEQ ID NO:11 (TF4). In one embodiment, the nucleic acid sequence comprises SEQ ID NO:14 (TF5). In one embodiment, the plant cell comprises an algae species. In one embodiment, the algae species comprises Chlamydomonas reinhardtii.

### DEFINITIONS

The term “upregulated” as used herein, should be interpreted in the most general sense possible. For example, a special type of molecule (i.e., for example, a nucleic acid) may be “upregulated” in a cell if it is produced at a level significantly and detectably higher (i.e., for example, between 1.5-10 fold) the natural expression rate. “Upregulation” of a molecule in a cell can be achieved via both traditional mutation and selection techniques and genetic manipulation methods.

The term “ectopic expression” as used herein, refers to any nucleic acid upregulation produced by an exogenous expression platform that is not natural to the plant cell (i.e., for example, a plant genome transfected by a vector).

To facilitate an understanding of the present invention, a number of terms and phrases as used herein are defined below:

The term “plant” is used in it broadest sense. It includes, but is not limited to; any species of woody, ornamental or decorative, crop or cereal, fruit or vegetable plant, and photosynthetic green algae (for example, Chlamydomonas reinhardtii). It also refers to a plurality of plant cells which are largely differentiated into a structure that is present at any stage of a plant's development. Such structures include, but are not limited to, a fruit, shoot, stem, leaf, flower petal, etc. The term “plant tissue” includes differentiated and undifferentiated tissues of plants including those present in roots, shoots, leaves, pollen, seeds and tumors, as well as cells in culture (for example, single cells, protoplasts, embryos, callus, etc.). Plant tissue may be in plants, in organ culture, tissue culture, or cell culture. The term “plant part” as used herein refers to a plant structure or a plant tissue.

The term “crop” or “crop plant” is used in its broadest sense. The term includes, but is not limited to, any species of plant or algae edible by humans or used as a feed for animals or used, or consumed by humans, or any plant or algae used in industry or commerce.

The term “oil-producing species” refers to plant species that produce and store triacylglycerol in specific organs, primarily in seeds. Such species include, but are not limited to, green algae (Chlamydomonas reinhardtii), soybean (Glycine max), rapeseed and canola (including Brassica napus and B. campestris), sunflower (Helianthus annus), cotton (Gossypium hirsutum), corn (Zea mays), cocoa (Theobroma cacao), safflower (Carthamus tinctorius), oil palm (Elaeis guineensis), coconut palm (Cocos nucifera), flax (Linum usitatissimum), castor (Ricinus communis) and peanut (Arachis hypogaea). The group also includes non-agronomic species which are useful in developing appropriate expression vectors such as tobacco, rapid cycling Brassica species, and Arabidopsis thaliana, and wild species which may be a source of unique fatty acids.

The term “Chlamydomonas” refers to a plant or plants from the genus Chlamydomonas. Non-limiting examples of Chlamydomonas include plants from the species C. reindardtii. The term also refers to C. reindardtii algae from which nucleic acid sequence SEQ ID NOs: 1-15 were isolated.

The term plant cell “compartments or organelles” is used in its broadest sense. The term includes, but is not limited to, the endoplasmic reticulum, Golgi apparatus, trans Golgi network, plastids, sarcoplasmic reticulum, glyoxysomes, mitochondrial, chloroplast, and nuclear membranes, and the like.

The term “host cell” refers to any cell capable of replicating and/or transcribing and/or translating a heterologous gene.

The terms “diacylglycerol” and “diglyceride” refer to a molecule comprising a glycerol backbone to which two acyl groups are esterified. Typically, the acyl groups are esterified to the sn-1 and sn-2 positions, although the acyl groups may also be esterified to the sn-1 and sn-3 positions, or to the sn-2 and sn-3 positions; the remaining position is unesterified and contains a hydroxyl group. This term may be represented by the abbreviation DAG.

The terms “triacylglycerol” and “triglyceride” refer to a molecule comprising a glycerol backbone to which three acyl groups are esterified. This term may be represented by the abbreviation TAG.

The term “long chain triacylglycerol” refers to a triacylglycerol in which all three acyl groups are long chain, or in other words each chain is a linear aliphatic chain of 6 carbons or greater in length (an acyl group may be referred to by the letter C followed by the number of carbons in the linear aliphatic chain, as, for example, C6 refers to an acyl group of 6 carbons in length). This term may be represented by the abbreviation LcTAG.

The terms “acetyl glyceride” and “acetyl triacylglycerol” and the like refer to a triglyceride to which at least one acetyl or related group is esterified to the glycerol backbone. A particular acetyl glyceride is denoted by the position(s) to which an acetyl or related group is esterified; thus, “sn-3-acetyl glyceride” or “1,2-diacyl-3-acetin” refers to triacylglycerol with an acetyl group at the sn-3 position. These terms may be represented by the abbreviation AcTAG.

An “acetyl” or “related group”, when used in reference to AcTAG, refers to an acyl moiety other than a long-chain acyl group esterified to TAG. The acyl moiety is any linear aliphatic chain of less than 6 carbons in length; it may or may not have side group chains or substituents. The acyl moiety may also be aromatic. Related group members include but are not limited to propionyl and butyryl groups, and aromatic groups such as benzoyl and cinnamoyl.

The term “diacylglycerol acyltransferase” refers to a polypeptide with the capacity to transfer an acyl group to a diacylglycerol substrate. Typically, a diacylglycerol acyltransferase transfers an acyl group to the sn-3 position of the diacylglycerol, though transfer to the sn-1 and sn-2 positions are also possible. The acyl substrate for the transferase is typically esterified to CoA; thus, the acyl substrate is typically acyl-CoA. The enzyme is therefore also referred to as an “diacylglycerol:acyl-CoA acyltransferase,” and in some particular embodiments, as an “acyl-CoA:sn-1,2-diacylglycerol acyltransferase,” and the like. The term may be referred to by the abbreviation DAGAT.

The term “diacylglycerol acetyltransferase” refers to a diacylglycerol acyltransferase polypeptide with a unique acyl group transfer specificity, such that the polypeptide is able to transfer an acetyl or related group to a diacylglycerol substrate, and such that the diacylglycerol acetyltransferase exhibits increased specificity for an acetyl or related group compared to a diacylglycerol acyltransferase obtained from a plant in which acetyl TAGs are not present, or are present in only trace amounts (in other words, less than about 1% of the total TAGs). The specificity may be determined by either in vivo or in vitro assays. From an in vivo assay, the specificity is the proportion of total TAGs that are AcTAGs, where the AcTAGs are synthesized by the presence of a heterologous diacylglycerol acetyltransferase. From an in vitro assay, the specificity is the activity of transfer of an acetyl or related group to a diacylglycerol, when the substrate is an acetyl-CoA or related group esterified to CoA. The increase in specificity of transferring an acetyl or related group for an AcDAGAT is at least about 1.5 times, or about 2 times, or about 5 times, or about 10 times, or about 20 times, or about 50 times, or about 100 times, or up to about 2000 times, the specificity of a DAGAT obtained from a plant in which acetyl TAGs are not present, or are present in only trace amounts. One standard DAGAT to which an AcDAGAT is compared, in order to determine specificity of transfer of an acetyl or related group, is a DAGAT obtained from Arabidopsis (AtDAGAT).

The acetyl or related group substrate of the transferase is typically esterified to CoA; thus, typical acetyl substrate include but are not limited to acetyl-CoA, propionyl-CoA, butyryl-CoA, benzoyl-CoA, or cinnamoyl-CoA, as described above. These CoA substrates are typically non-micellar acyl-CoAs, or possess high critical micelle concentrations (CMCs), in that they form micelles at relatively high concentrations when compared to the CMCs of long chain acyl-CoAs.

The diacylglycerol substrate of AcDAGAT is typically a long chain diacylglycerol, although other groups are also contemplated. The acyl (or other) groups are esterified to the sn-1 and sn-2 positions, although the acyl groups may also be esterified to the sn-1 and sn-3 positions, or to the sn-2 and sn-3 positions.

Thus, the enzyme is also referred to as an “diacylglycerol:acetyl-CoA acetyltransferase,” or in particular embodiments, as an “acetyl-CoA:sn-1,2-diacylglycerol acetyltransferase” and the like. This term may be referred to by the abbreviation AcDAGAT, indicating an activity of increased specificity for transfer of acetyl or related groups

The terms “Chlamydomonas” and “Chlamydomonas-like” when used in reference to a DAGAT refer to a DAGAT obtained from Chlamydomonas reinhardtii or with a substrate specificity that is similar to a DAGAT obtained from Chlamydomonas reinhardtii. The term may be referred to by the abbreviation, “ChDAGAT,” indicating an enzyme obtained from Chlamydomonas reinhardtii, or from the genus Chlamydomonas, or from a closely related plant family, or an enzyme which has an amino acid sequence with a high degree of similarity to or identity with a DAGAT obtained from Chlamydomonas reinhardtii. By “high degree of similarity” it is meant that it is more closely related to ChDAGAT than to AtDAGAT by BLAST scores or other amino acid sequence comparison/alignment software programs.

The term “substrate specificity” refers to the range of substrates that an enzyme will act upon to produce a product.

The term “competes for binding” is used in reference to a first polypeptide with enzymatic activity which binds to the same substrate as does a second polypeptide with enzymatic activity, where the second polypeptide is variant of the first polypeptide or a related or dissimilar polypeptide. The efficiency (for example, kinetics or thermodynamics) of binding by the first polypeptide may be the same as or greater than or less than the efficiency substrate binding by the second polypeptide. For example, the equilibrium binding constants (K_{D}) for binding to the substrate may be different for the two polypeptides.

The terms “protein” and “polypeptide” refer to compounds comprising amino acids joined via peptide bonds and are used interchangeably.

As used herein, “amino acid sequence” refers to an amino acid sequence of a protein molecule. “Amino acid sequence” and like terms, such as “polypeptide” or “protein,” are not meant to limit the amino acid sequence to the complete, native amino acid sequence associated with the recited protein molecule. Furthermore, an “amino acid sequence” can be deduced from the nucleic acid sequence encoding the protein.

The term “portion” when used in reference to a protein (as in “a portion of a given protein”) refers to fragments of that protein. The fragments may range in size from four amino acid residues to the entire amino sequence minus one amino acid.

The term “homology” when used in relation to amino acids refers to a degree of similarity or identity. There may be partial homology or complete homology (in other words, identity). “Sequence identity” refers to a measure of relatedness between two or more proteins, and is given as a percentage with reference to the total comparison length. The identity calculation takes into account those amino acid residues that are identical and in the same relative positions in their respective larger sequences. Calculations of identity may be performed by algorithms contained within computer programs.

The term “chimera” when used in reference to a polypeptide refers to the expression product of two or more coding sequences obtained from different genes, that have been cloned together and that, after translation, act as a single polypeptide sequence. Chimeric polypeptides are also referred to as “hybrid” polypeptides. The coding sequences include those obtained from the same or from different species of organisms.

The term “fusion” when used in reference to a polypeptide refers to a chimeric protein containing a protein of interest joined to an exogenous protein fragment (the fusion partner). The fusion partner may serve various functions, including enhancement of solubility of the polypeptide of interest, as well as providing an “affinity tag” to allow purification of the recombinant fusion polypeptide from a host cell or from a supernatant or from both. If desired, the fusion partner may be removed from the protein of interest after or during purification.

The term “homolog” or “homologous” when used in reference to a polypeptide refers to a high degree of sequence identity between two polypeptides, or to a high degree of similarity between the three-dimensional structure or to a high degree of similarity between the active site and the mechanism of action. In a preferred embodiment, a homolog has a greater than 60% sequence identity, and more preferable greater than 75% sequence identity, and still more preferably greater than 90% sequence identity, with a reference sequence.

The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties (for example, replacement of leucine with isoleucine). More rarely, a variant may have “non-conservative” changes (for example, replacement of a glycine with a tryptophan). Similar minor variations may also include amino acid deletions or insertions (in other words, additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

The term “gene” refers to a nucleic acid (for example, DNA or RNA) sequence that comprises coding sequences necessary for the production of RNA, or a polypeptide or its precursor (for example, proinsulin). A functional polypeptide can be encoded by a full length coding sequence or by any portion of the coding sequence as long as the desired activity or functional properties (for example, enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage and polyadenylation.

The term “heterologous gene” refers to a gene encoding a factor that is not in its natural environment (in other words, has been altered by the hand of man). For example, a heterologous gene includes a gene from one species introduced into another species. A heterologous gene also includes a gene native to an organism that has been altered in some way (for example, mutated, added in multiple copies, linked to a non-native promoter or enhancer sequence, etc.). Heterologous genes may comprise plant gene sequences that comprise cDNA forms of a plant gene; the cDNA sequences may be expressed in either a sense (to produce mRNA) or anti-sense orientation (to produce an anti-sense RNA transcript that is complementary to the mRNA transcript). Heterologous genes are distinguished from endogenous plant genes in that the heterologous gene sequences are typically joined to nucleotide sequences comprising regulatory elements such as promoters that are not found naturally associated with the gene for the protein encoded by the heterologous gene or with plant gene sequences in the chromosome, or are associated with portions of the chromosome not found in nature (for example, genes expressed in loci where the gene is not normally expressed).

The term “oligonucleotide” refers to a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

The term “an oligonucleotide having a nucleotide sequence encoding a gene” or “a nucleic acid sequence encoding” a specified polypeptide refers to a nucleic acid sequence comprising the coding region of a gene or in other words the nucleic acid sequence which encodes a gene product. The coding region may be present in either a cDNA, genomic DNA or RNA form. When present in a DNA form, the oligonucleotide may be single-stranded (in other words, the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

The terms “complementary” and “complementarity” refer to polynucleotides (in other words, a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” when used in relation to nucleic acids refers to a degree of complementarity. There may be partial homology or complete homology (in other words, identity). “Sequence identity” refers to a measure of relatedness between two or more nucleic acids, and is given as a percentage with reference to the total comparison length. The identity calculation takes into account those nucleotide residues that are identical and in the same relative positions in their respective larger sequences. Calculations of identity may be performed by algorithms contained within computer programs such as “GAP” (Genetics Computer Group, Madison, Wis.) and “ALIGN” (DNAStar, Madison, Wis.). A partially complementary sequence is one that at least partially inhibits (or competes with) a completely complementary sequence from hybridizing to a target nucleic acid is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (in other words, the hybridization) of a sequence that is completely homologous to a target under conditions of low stringency. This is not to say that conditions of low stringency are such that non-specific binding is permitted; low stringency conditions require that the binding of two sequences to one another be a specific (in other words, selective) interaction. The absence of non-specific binding may be tested by the use of a second target which lacks even a partial degree of complementarity (for example, less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe which can hybridize to either or both strands of the double-stranded nucleic acid sequence under conditions of low stringency as described infra.

Low stringency conditions when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharmacia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed. Numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (for example, the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (for example, increasing the temperature of the hybridization and/or wash steps, the use of formamide in the hybridization solution, etc.).

“High stringency” conditions are used in reference to nucleic acid hybridization comprise conditions equivalent to binding or hybridization at 42.degree. C. in a solution consisting of 5.times.SSPE (43.8 g/l NaCl, 6.9 g/l NaH.sub.2PO.sub.4.H.sub.2O and 1.85 g/l EDTA, pH adjusted to 7.4 with NaOH), 0.5% SDS, 5.times. Denhardt's reagent and 100 μg/ml denatured salmon sperm DNA followed by washing in a solution comprising 0.1.times.SSPE, 1.0% SDS at 42.degree. C. when a probe of about 500 nucleotides in length is employed.

The term “substantially homologous”, when used in reference to a double-stranded nucleic acid sequence such as a cDNA or genomic clone, refers to any probe that can hybridize to either or both strands of the double-stranded nucleic acid sequence under conditions of low to high stringency as described above.

The term “substantially homologous”, when used in reference to a single-stranded nucleic acid sequence, refers to any probe that can hybridize (in other words, it is the complement of) the single-stranded nucleic acid sequence under conditions of low to high stringency as described above.

The term “hybridization” refers to the pairing of complementary nucleic acids. Hybridization and the strength of hybridization (in other words, the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

The term “Tₘ” refers to the “melting temperature” of a nucleic acid. The melting temperature is the temperature at which a population of double-stranded nucleic acid molecules becomes half dissociated into single strands. The equation for calculating the Tₙ, of nucleic acids may be calculated by: Tₘ=81.5.+−.0.41(% G+C), when a nucleic acid is in aqueous solution at 1 M NaCl (See for example, Anderson and Young, Quantitative Filter Hybridization (1985) in Nucleic Acid Hybridization). Other references include more sophisticated computations that take structural as well as sequence characteristics into account for the calculation of Tₘ.

As used herein the term “stringency” refers to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “low” stringency are often required with nucleic acids that are derived from organisms that are genetically diverse, as the frequency of complementary sequences is usually less.

“Amplification” is a special case of nucleic acid replication involving template specificity. It is to be contrasted with non-specific template replication (in other words, replication that is template-dependent but not dependent on a specific template). Template specificity is here distinguished from fidelity of replication (in other words, synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Qβreplicase, MDV-1 RNA is the specific template for the replicase (Kacian et al. (1972) Proc. Natl. Acad. Sci. USA, 69:3038). Other nucleic acid will not be replicated by this amplification enzyme. Similarly, in the case of T7 RNA polymerase, this amplification enzyme has a stringent specificity for its own promoters (Chamberlin et al. (1970) Nature, 228:227). In the case of T4 DNA ligase, the enzyme will not ligate the two oligonucleotides or polynucleotides, where there is a mismatch between the oligonucleotide or polynucleotide substrate and the template at the ligation junction (Wu and Wallace (1989) Genomics, 4:560). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.) (1989) PCR Technology, Stockton Press).

The term “amplifiable nucleic acid” refers to nucleic acids that may be amplified by any amplification method. It is contemplated that “amplifiable nucleic acid” will usually comprise “sample template.”

The term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

The term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (in other words, in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The term “polymerase chain reaction” (“PCR”) refers to the method of K. B. Mullis U.S. Pat. Nos. 4,683,195, 4,683,202, and 4,965,188, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (in other words, denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.” With PCR, it is possible to amplify a single copy of a specific target sequence in genomic DNA to a level detectable by several different methodologies (for example, hybridization with a labeled probe; incorporation of biotinylated primers followed by avidin-enzyme conjugate detection; incorporation of .sup.32P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

The terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

The term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

The term “reverse-transcriptase” or “RT-PCR” refers to a type of PCR where the starting material is mRNA. The starting mRNA is enzymatically converted to complementary DNA or “cDNA” using a reverse transcriptase enzyme. The cDNA is then used as a “template” for a “PCR” reaction.

The term “RACE” refers to Rapid Amplification of cDNA Ends.

The term “gene expression” refers to the process of converting genetic information encoded in a gene into RNA (for example, mRNA, rRNA, tRNA, or snRNA) through “transcription” of the gene (in other words, via the enzymatic action of an RNA polymerase), and into protein, through “translation” of mRNA. Gene expression can be regulated at many stages in the process. “Up-regulation” or “activation” refers to regulation that increases the production of gene expression products (in other words, RNA or protein), while “down-regulation” or “repression” refers to regulation that decrease production. Molecules (for example, transcription factors) that are involved in up-regulation or down-regulation are often called “activators” and “repressors,” respectively.

The terms “in operable combination”, “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element that controls some aspect of the expression of nucleic acid sequences. For example, a promoter is a regulatory element that facilitates the initiation of transcription of an operably linked coding region. Other regulatory elements are splicing signals, polyadenylation signals, termination signals, etc.

The terms “promoter” and “enhancer” as used herein are examples of transcriptional control signals. Promoters and enhancers comprise short arrays of DNA sequences that interact specifically with cellular proteins involved in transcription (Maniatis, et al., Science 236:1237, 1987). Promoter and enhancer elements have been isolated from a variety of eukaryotic sources including genes in yeast, algae insect, mammalian and plant cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types (for review, see Voss, et al., Trends Biochem. Sci., 11:287, 1986; and Maniatis, et al., supra 1987).

The terms “promoter element,” “promoter,” or “promoter sequence” as used herein, refer to a DNA sequence that is located at the 5′ end (in other words precedes) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA. Promoters may be tissue specific or cell specific.

The term “tissue specific” as it applies to a promoter refers to a promoter that is capable of directing selective expression of a nucleotide sequence of interest to a specific type of tissue (for example, seeds) in the relative absence of expression of the same nucleotide sequence of interest in a different type of tissue (for example, leaves). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (for example, detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected.

The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, for example, immunohistochemical staining. Briefly, tissue sections are embedded in paraffin, and paraffin sections are reacted with a primary antibody that is specific for the polypeptide product encoded by the nucleotide sequence of interest whose expression is controlled by the promoter. A labeled (for example, peroxidase conjugated) secondary antibody that is specific for the primary antibody is allowed to bind to the sectioned tissue and specific binding detected (for example, with avidin/biotin) by microscopy.

The term “constitutive” when made in reference to a promoter means that the promoter is capable of directing transcription of an operably linked nucleic acid sequence in the absence of a stimulus (for example, heat shock, chemicals, light, etc.). Typically, constitutive promoters are capable of directing expression of a transgene in substantially any cell and any tissue. Exemplary constitutive plant promoters include, but are not limited to SD Cauliflower Mosaic Virus (CaMV SD; see for example, U.S. Pat. No. 5,352,605, incorporated herein by reference), mannopine synthase, octopine synthase (ocs), superpromoter (see for example, WO 95/14098), and ubi3 (see for example, Garbarino and Belknap (1994) Plant Mol. Biol. 24:119-127) promoters. Such promoters have been used successfully to direct the expression of heterologous nucleic acid sequences in transformed plant tissue.

The term “regulatable” or “inducible”, when made in reference to a promoter is one that is capable of directing a level of transcription of an operably linked nuclei acid sequence in the presence of a stimulus (for example, heat shock, chemicals, light, etc.) which is different from the level of transcription of the operably linked nucleic acid sequence in the absence of the stimulus.

An “endogenous” enhancer or promoter is one that is naturally linked with a given gene in the genome.

An “exogenous”, “ectopic” or “heterologous” enhancer or promoter is one that is placed in juxtaposition to a gene by means of genetic manipulation (in other words, molecular biological techniques) such that transcription of the gene is directed by the linked enhancer or promoter. For example, an endogenous promoter in operable combination with a first gene can be isolated, removed, and placed in operable combination with a second gene, thereby making it a “heterologous promoter” in operable combination with the second gene. A variety of such combinations are contemplated (for example, the first and second genes can be from the same species, or from different species.

The presence of “splicing signals” on an expression vector often results in higher levels of expression of the recombinant transcript in eukaryotic host cells. Splicing signals mediate the removal of introns from the primary RNA transcript and consist of a splice donor and acceptor site (Sambrook, et al. (1989) Molecular Cloning: A Laboratory Manual, 2nd ed., Cold Spring Harbor Laboratory Press, New York, pp. 16.7-16.8). A commonly used splice donor and acceptor site is the splice junction from the 16S RNA of SV40. Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly(A) site” or “poly(A) sequence” as used herein denotes a DNA sequence which directs both the termination and polyadenylation of the nascent RNA transcript. Efficient polyadenylation of the recombinant transcript is desirable, as transcripts lacking a poly(A) tail are unstable and are rapidly degraded. The poly(A) signal utilized in an expression vector may be “heterologous” or “endogenous.” An endogenous poly(A) signal is one that is found naturally at the 3′ end of the coding region of a given gene in the genome. A heterologous poly(A) signal is one which has been isolated from one gene and positioned 3′ to another gene. A commonly used heterologous poly(A) signal is the SV40 poly(A) signal. The SV40 poly(A) signal is contained on a 237 by BamHI/BclI restriction fragment and directs both termination and polyadenylation (Sambrook, supra, at 16.6-16.7).

The term “selectable marker” refers to a gene which encodes an enzyme having an activity that confers resistance to an antibiotic or drug upon the cell in which the selectable marker is expressed, or which confers expression of a trait which can be detected (for example luminescence or fluorescence). Selectable markers may be “positive” or “negative.” Examples of positive selectable markers include the neomycin phosphotransferase (NPTII) gene that confers resistance to G418 and to kanamycin, and the bacterial hygromycin phosphotransferase gene (hyg), which confers resistance to the antibiotic hygromycin. Negative selectable markers encode an enzymatic activity whose expression is cytotoxic to the cell when grown in an appropriate selective medium. For example, the HSV-tk gene is commonly used as a negative selectable marker. Expression of the HSV-tk gene in cells grown in the presence of gancyclovir or acyclovir is cytotoxic; thus, growth of cells in selective medium containing gancyclovir or acyclovir selects against cells capable of expressing a functional HSV TK enzyme.

The term “vector” as used herein, refers to any nucleic acid molecule that transfers DNA segment(s) from one cell to another. The term “vehicle” is sometimes used interchangeably with “vector.”

The terms “expression vector” or “expression cassette” as used herein, refer to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The term “transfection”, as used herein, refers to the introduction of foreign DNA into cells. Transfection may be accomplished by a variety of means known to the art including calcium phosphate-DNA co-precipitation, DEAE-dextran-mediated transfection, polybrene-mediated transfection, glass beads, electroporation, microinjection, liposome fusion, lipofection, protoplast fusion, viral infection, biolistics (in other words, particle bombardment) and the like.

The term “Agrobacterium” refers to a soil-borne, Gram-negative, rod-shaped phytopathogenic bacterium that causes crown gall. The term “Agrobacterium” includes, but is not limited to, the strains Agrobacterium tumefaciens, (which typically causes crown gall in infected plants), and Agrobacterium rhizogens (which causes hairy root disease in infected host plants). Infection of a plant cell with Agrobacterium generally results in the production of opines (for example, nopaline, agropine, octopine etc.) by the infected cell. Thus, Agrobacterium strains which cause production of nopaline (for example, strain LBA4301, C58, A208, GV3101) are referred to as “nopaline-type” Agrobacteria; Agrobacterium strains which cause production of octopine (for example, strain LBA4404, Ach5, B6) are referred to as “octopine-type” Agrobacteria; and Agrobacterium strains which cause production of agropine (for example, strain EHA105, EHA101, A281) are referred to as “agropine-type” Agrobacteria.

The terms “bombarding, “bombardment,” and “biolistic bombardment” refer to the process of accelerating particles towards a target biological sample (for example, cell, tissue, etc.) to effect wounding of the cell membrane of a cell in the target biological sample and/or entry of the particles into the target biological sample. Methods for biolistic bombardment are known in the art (for example, U.S. Pat. No. 5,584,807, the contents of which are incorporated herein by reference), and are commercially available (for example, the helium gas-driven microprojectile accelerator (PDS-1000/He, BioRad).

The term “microwounding” when made in reference to plant tissue refers to the introduction of microscopic wounds in that tissue. Microwounding may be achieved by, for example, particle bombardment as described herein.

The term “transgenic” when used in reference to a plant or fruit or seed (in other words, a “transgenic plant” or “transgenic fruit” or a “transgenic seed”) refers to a plant or fruit or seed that contains at least one heterologous gene in one or more of its cells. The term “transgenic plant material” refers broadly to a plant, a plant structure, a plant tissue, a plant seed or a plant cell that contains at least one heterologous gene in one or more of its cells.

The terms “transformants” or “transformed cells” include the primary transformed cell and cultures derived from that cell without regard to the number of transfers. All progeny may not be precisely identical in DNA content, due to deliberate or inadvertent mutations. Mutant progeny that have the same functionality as screened for in the originally transformed cell are included in the definition of transformants.

The term “wild-type”, “native”, or “natural” when made in reference to a gene or protein that has the characteristics of a gene or protein isolated from a naturally occurring source. The term “wild-type” when made in reference to a gene or protein product refers to a gene or protein product that has the characteristics of a gene or protein product isolated from a naturally occurring source. A wild-type gene or protein is that which is most frequently observed in a population and is thus arbitrarily designated the “normal” form. In contrast, the term “modified” or “mutant” when made in reference to a gene, gene product, or protein refers, respectively, to a gene, gene product, or protein which displays modifications in sequence and/or functional properties (in other words, altered characteristics) when compared to the wild-type gene or gene product. It is noted that naturally-occurring mutants can be isolated; these are identified by the fact that they have altered characteristics when compared to the wild-type gene, gene product or protein.

The term “antisense” refers to a deoxyribonucleotide sequence whose sequence of deoxyribonucleotide residues is in reverse 5′ to 3′ orientation in relation to the sequence of deoxyribonucleotide residues in a sense strand of a DNA duplex. A “sense strand” of a DNA duplex refers to a strand in a DNA duplex that is transcribed by a cell in its natural state into a “sense mRNA.” Thus an “antisense” sequence is a sequence having the same sequence as the non-coding strand in a DNA duplex. The term “antisense RNA” refers to a RNA transcript that is complementary to all or part of a target primary transcript or mRNA and that blocks the expression of a target gene by interfering with the processing, transport and/or translation of its primary transcript or mRNA. The complementarity of an antisense RNA may be with any part of the specific gene transcript, in other words, at the 5′ non-coding sequence, 3′ non-coding sequence, introns, or the coding sequence. In addition, as used herein, antisense RNA may contain regions of ribozyme sequences that increase the efficacy of antisense RNA to block gene expression. “Ribozyme” refers to a catalytic RNA and includes sequence-specific endoribonucleases. “Antisense inhibition” refers to the production of antisense RNA transcripts capable of preventing the expression of the target protein.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “target RNA molecule” refers to an RNA molecule to which at least one strand of the short double-stranded region of an siRNA is homologous or complementary. Typically, when such homology or complementary is about 100%, the siRNA is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of siRNA. Such targets include unprocessed mRNA, ribosomal RNA, and viral RNA genomes.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “posttranscriptional gene silencing” or “PTGS” refers to silencing of gene expression in plants after transcription, and appears to involve the specific degradation of mRNAs synthesized from gene repeats.

The term “overexpression” refers to the production of a gene product in transgenic organisms that exceeds levels of production in normal or non-transformed organisms.

The term “cosuppression” refers to the expression of a foreign gene that has substantial homology to an endogenous gene resulting in the suppression of expression of both the foreign and the endogenous gene.

The term “altered levels” refers to the production of gene product(s) in transgenic organisms in amounts or proportions that differ from that of normal or non-transformed organisms.

The term “recombinant” when made in reference to a nucleic acid molecule refers to a nucleic acid molecule that is comprised of segments of nucleic acid joined together by means of molecular biological techniques.

The term “recombinant” when made in reference to a protein or a polypeptide refers to a protein molecule that is expressed using a recombinant nucleic acid molecule.

The terms “Southern blot analysis” and “Southern blot” and “Southern” refer to the analysis of DNA on agarose or acrylamide gels in which DNA is separated or fragmented according to size followed by transfer of the DNA from the gel to a solid support, such as nitrocellulose or a nylon membrane. The immobilized DNA is then exposed to a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al. (1989) Molecular Cloning: A Laboratory Manual, Cold Spring Harbor Press, NY, pp 9.31-9.58).

The term “Northern blot analysis” and “Northern blot” and “Northern” as used herein refer to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al. (1989) supra, pp 7.39-7.52).

The terms “Western blot analysis” and “Western blot” and “Western” refers to the analysis of protein(s) (or polypeptides) immobilized onto a support such as nitrocellulose or a membrane. A mixture comprising at least one protein is first separated on an acrylamide gel, and the separated proteins are then transferred from the gel to a solid support, such as nitrocellulose or a nylon membrane. The immobilized proteins are exposed to at least one antibody with reactivity against at least one antigen of interest. The bound antibodies may be detected by various methods, including the use of radiolabeled antibodies.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. For example, a given DNA sequence (for example, a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a plant DAGAT includes, by way of example, such nucleic acid in cells ordinarily expressing a DAGAT, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (in other words, the oligonucleotide may single-stranded), but may contain both the sense and anti-sense strands (in other words, the oligonucleotide may be double-stranded).

The term “purified” refers to molecules, either nucleic or amino acid sequences that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated. The term “purified” or “to purify” also refer to the removal of contaminants from a sample. The removal of contaminating proteins results in an increase in the percent of polypeptide of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “sample” is used in its broadest sense. In one sense it can refer to a plant cell or tissue. In another sense, it is meant to include a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

### BRIEF DESCRIPTION OF THE FIGURES

FIG. 1 depicts one embodiment of a transcription factor nucleic acid genomic sequence (SEQ ID NO:1), termed TF1: Protein ID: 159133; Location: Chlre31scaffold-38:677592-680772

FIG. 2 depicts one embodiment of a transcript nucleic acid sequence (SEQ ID NO:2) within SEQ ID NO:1.

FIG. 3 depicts one embodiment of a regulatory protein (SEQ ID NO:3) encoded by SEQ ID NO:2).

FIG. 4 depicts one embodiment of a transcription factor nucleic acid genomic sequence (SEQ ID NO:4), termed TF2: Assigned name: ESTEXT-FGENESH2-KG.C-370006; Protein ID: 185094; Location: Chlre3/scaffold-37:206092-208118.

FIG. 5 depicts one embodiment of a transcript nucleic acid sequence (SEQ ID NO:5) within SEQ ID NO:4.

FIG. 6 depicts one embodiment of a regulatory protein (SEQ ID NO:6) encoded by SEQ ID NO:5).

FIG. 7 depicts one embodiment of a transcription factor nucleic acid genomic sequence (SEQ ID NO:7), termed TF3: Assigned name: ESTEXT_FGENESH2_KG.C_{—}230055; Protein ID: 184359; Location: Chlre3/scaffold_{—}23: 1489894-1493691

FIG. 8 depicts one embodiment of a transcript nucleic acid sequence (SEQ ID NO:8) within SEQ ID NO:7.

FIG. 9 depicts one embodiment of a regulatory protein (SEQ ID NO:9) encoded by SEQ ID NO:8).

FIG. 10 depicts one embodiment of a transcription factor nucleic acid genomic sequence (SEQ ID NO:10), termed TF4: Assigned name: E-GWW.9.18.1; Protein ID: 114109; Location: Chlre31scaffold-9:2 101408-2106081.

FIG. 11 depicts one embodiment of a transcript nucleic acid sequence (SEQ ID NO:11) within SEQ ID NO:10.

FIG. 12 depicts one embodiment of a regulatory protein (SEQ ID NO:12) encoded by SEQ ID NO:11).

FIG. 13 depicts one embodiment of a transcription factor nucleic acid genomic sequence (SEQ ID NO:13), termed TF5: Assigned name: ACEGS-KG.SCAFFOLD-1000253; Protein ID: 157388; Location: Chlre3/scaffold-1:4 192945-4 194641.

FIG. 14 depicts one embodiment of a transcript nucleic acid sequence (SEQ ID NO:14) within SEQ ID NO:13.

FIG. 15 depicts one embodiment of a regulatory protein (SEQ ID NO:15) encoded by SEQ ID NO:14).

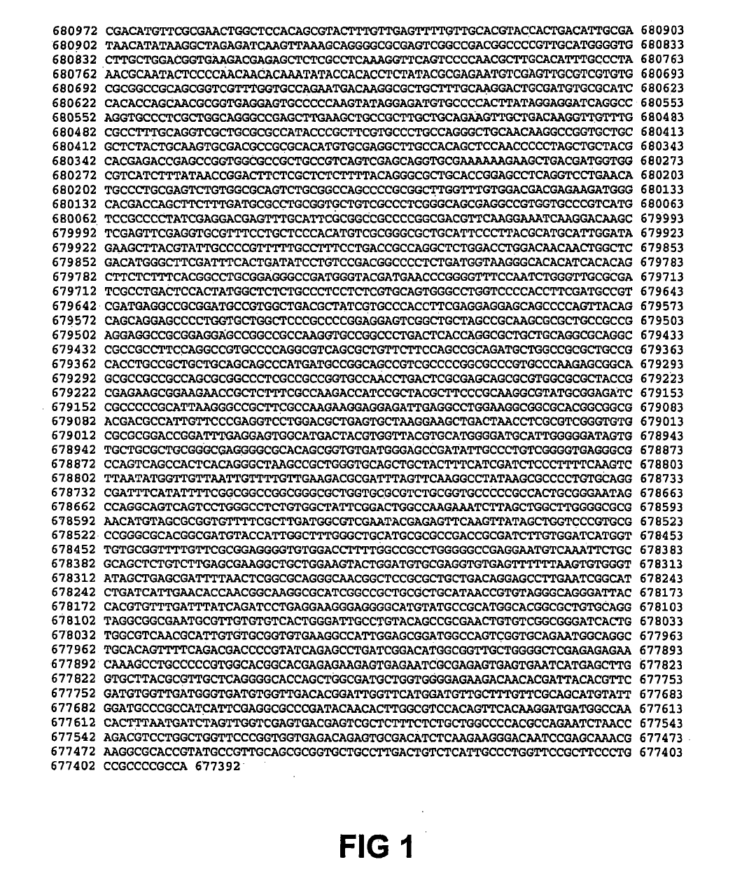

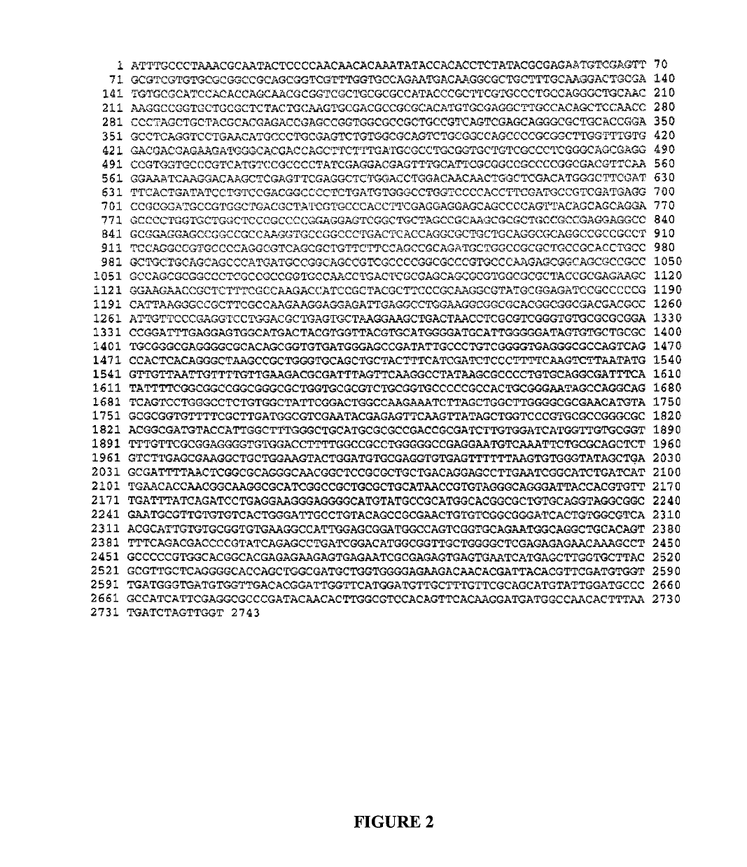

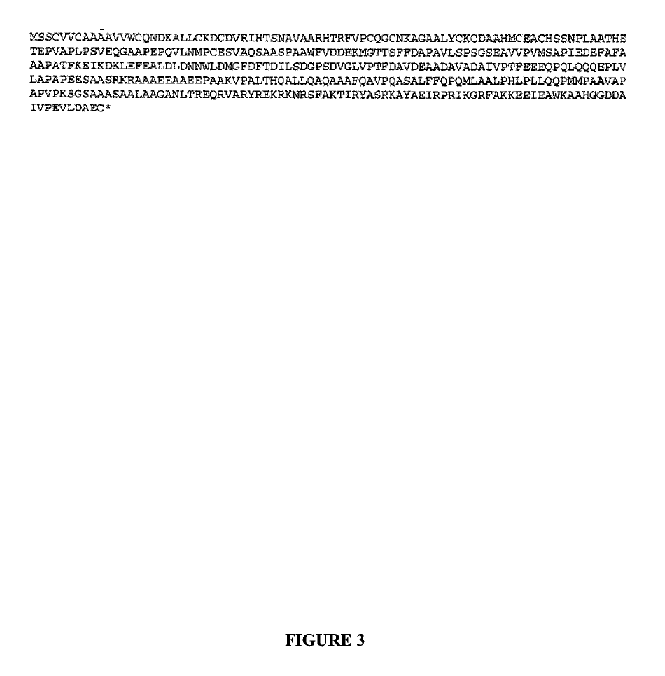

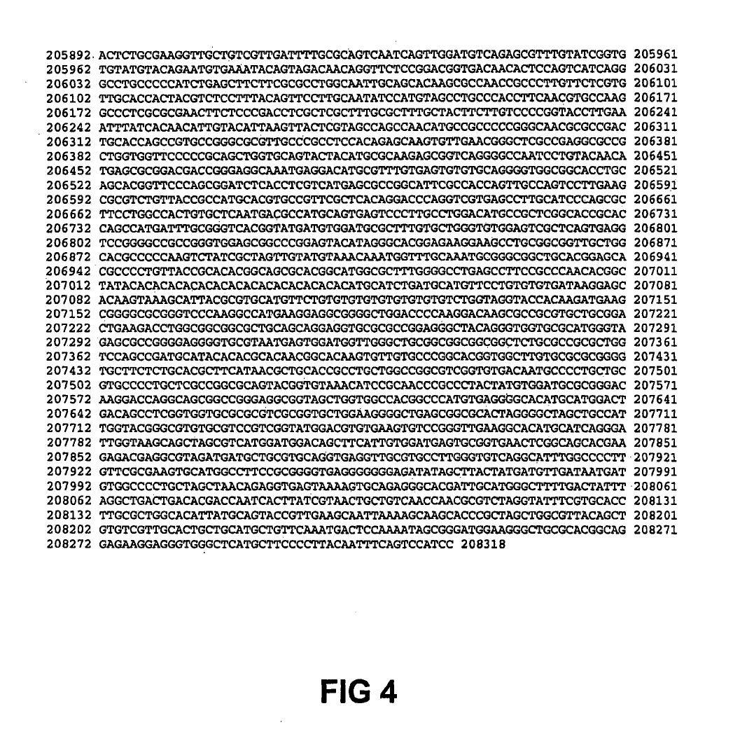

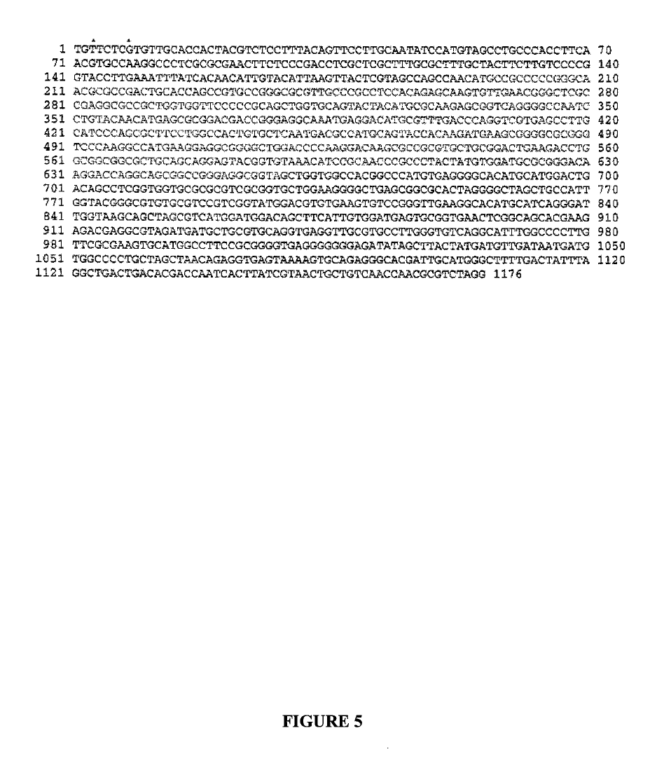

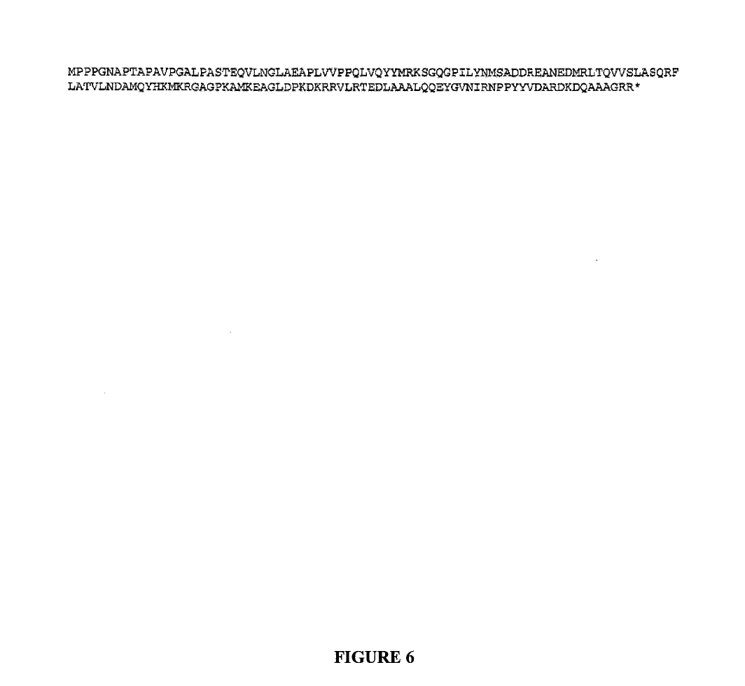

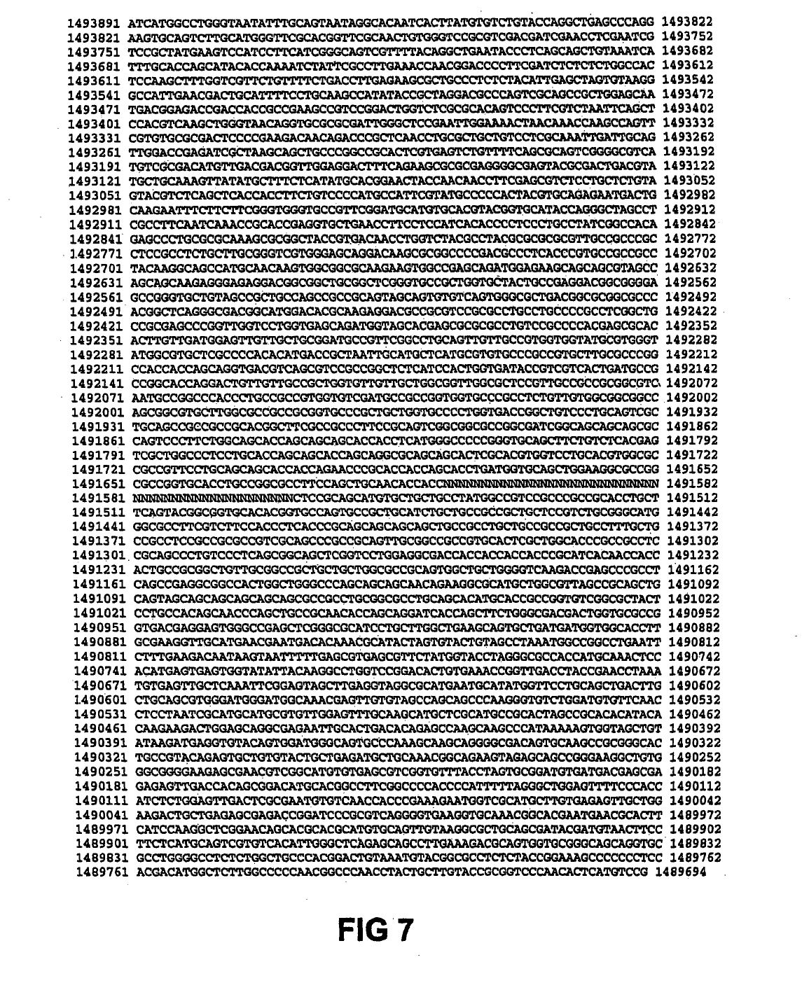

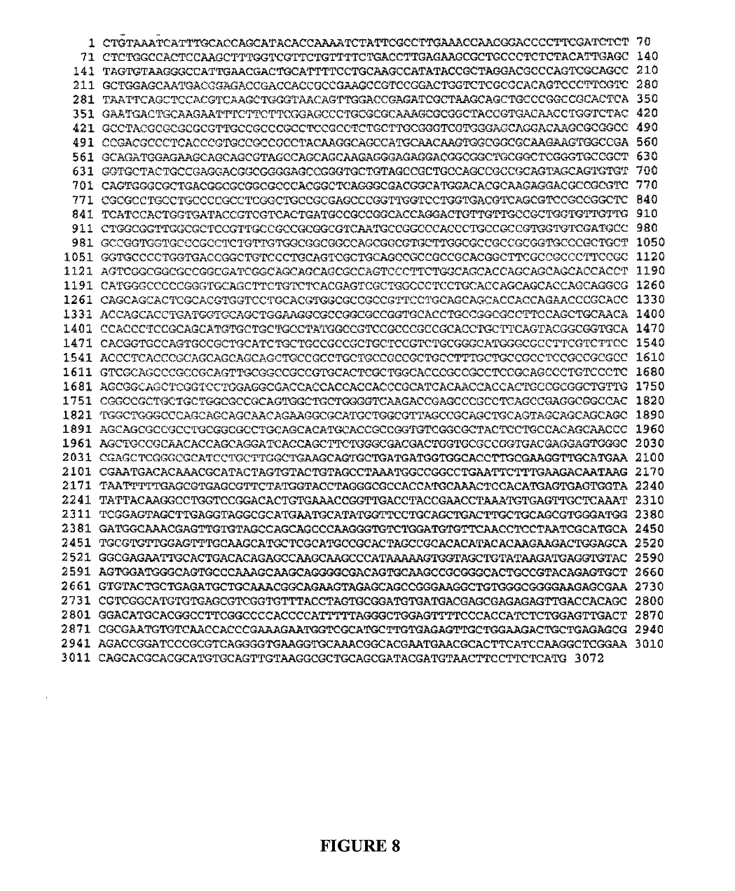

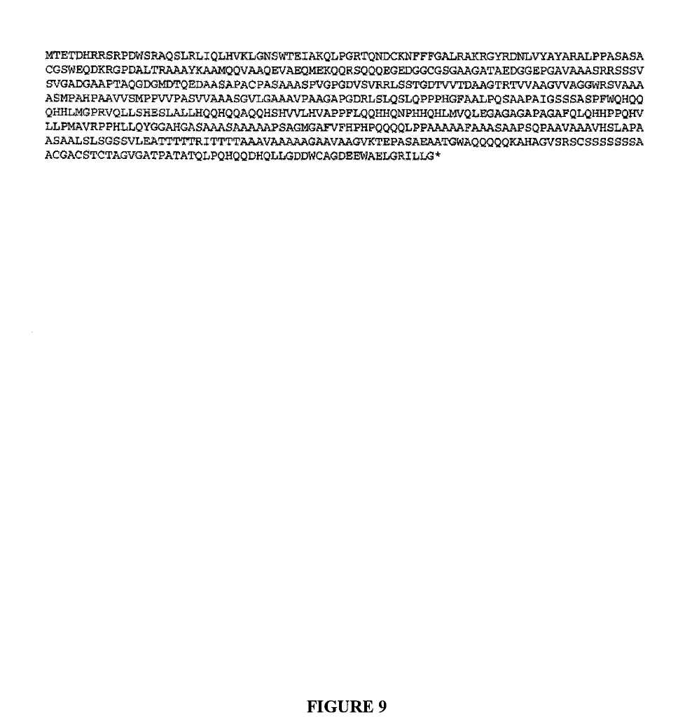

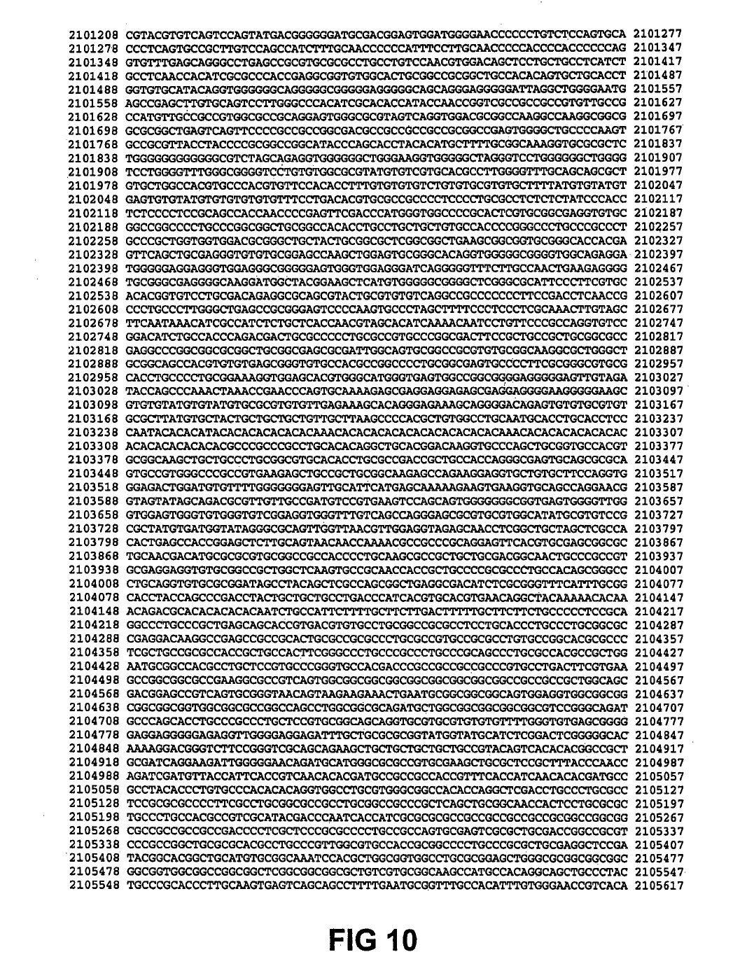

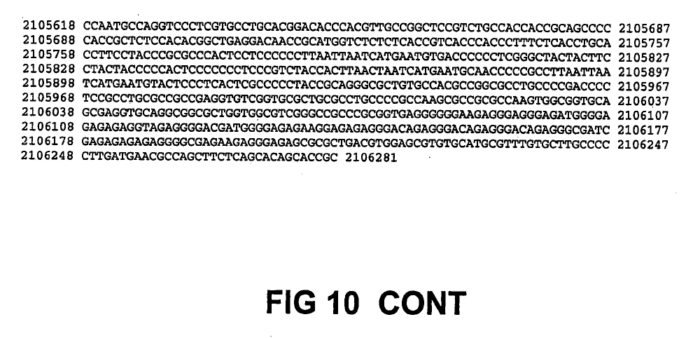

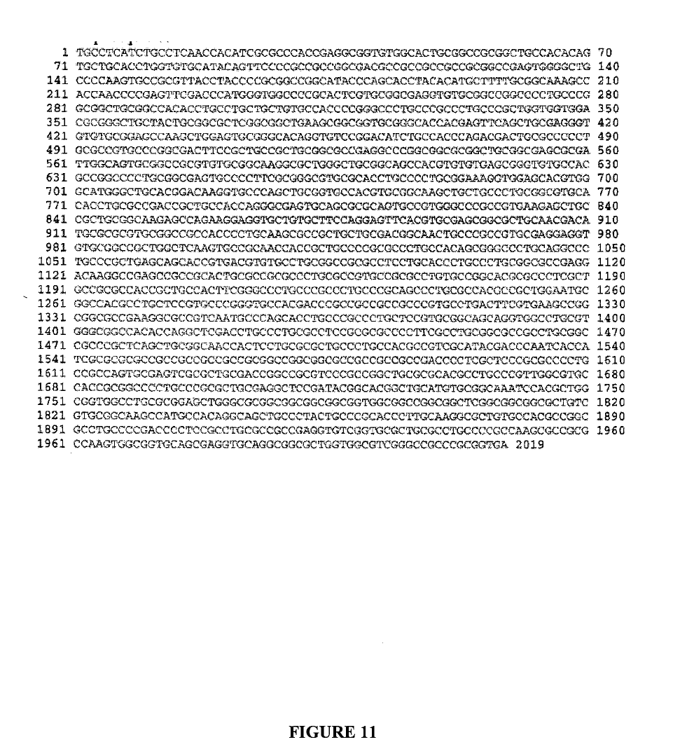

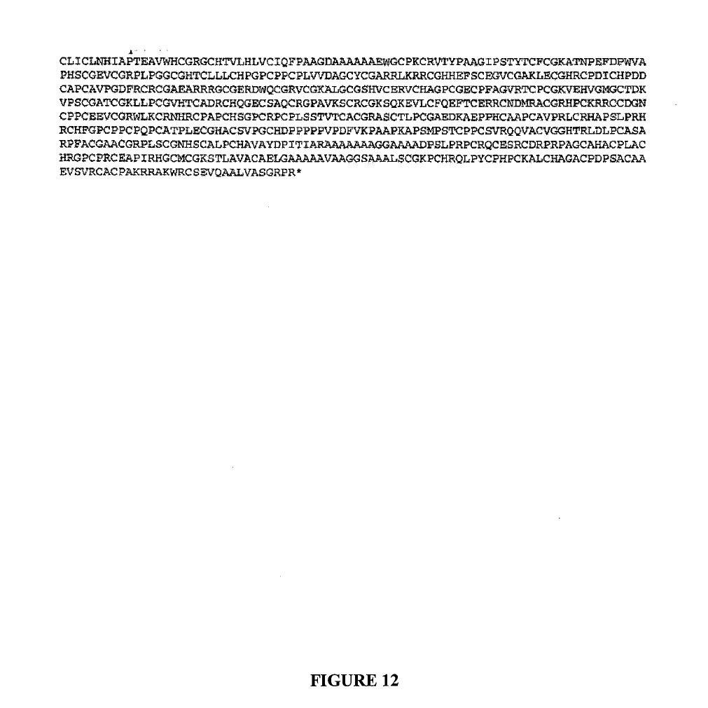

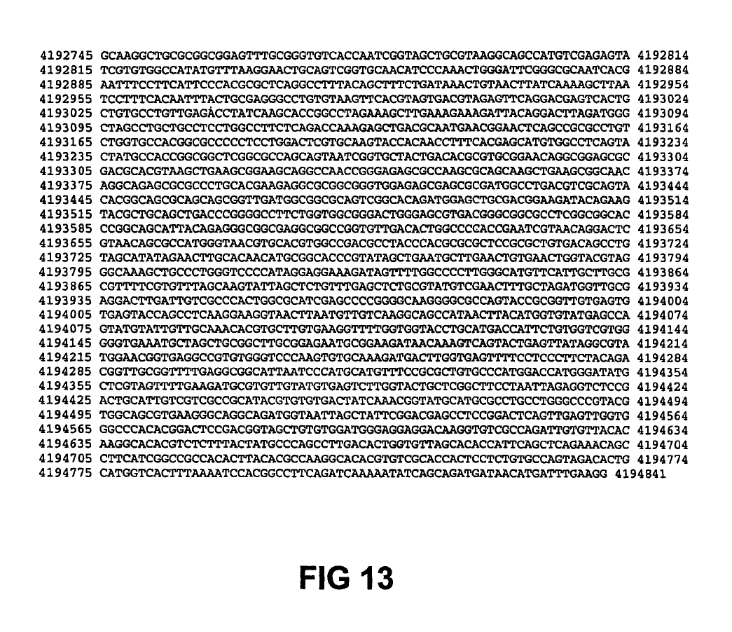

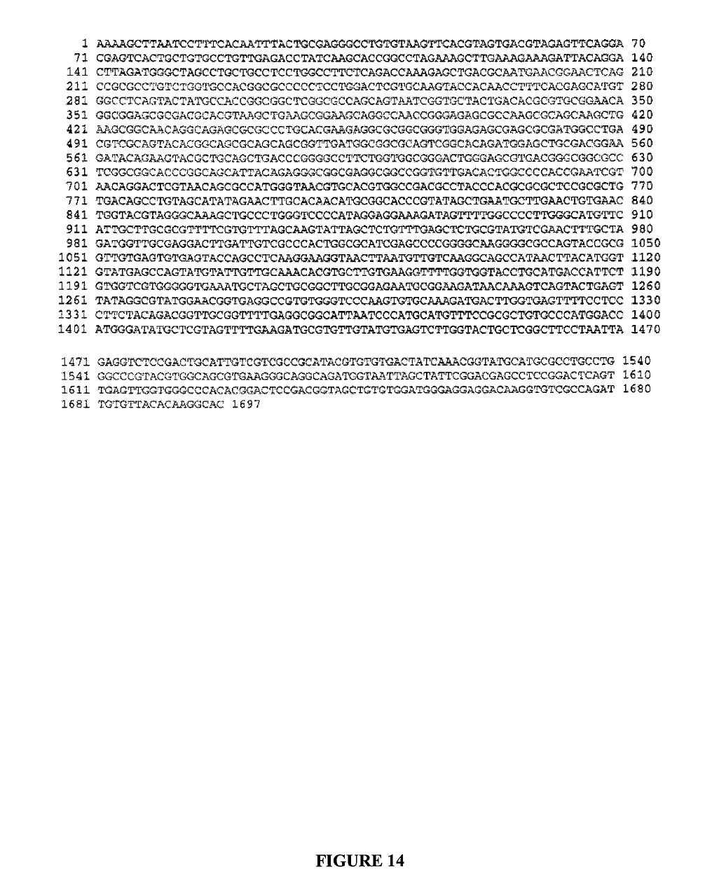

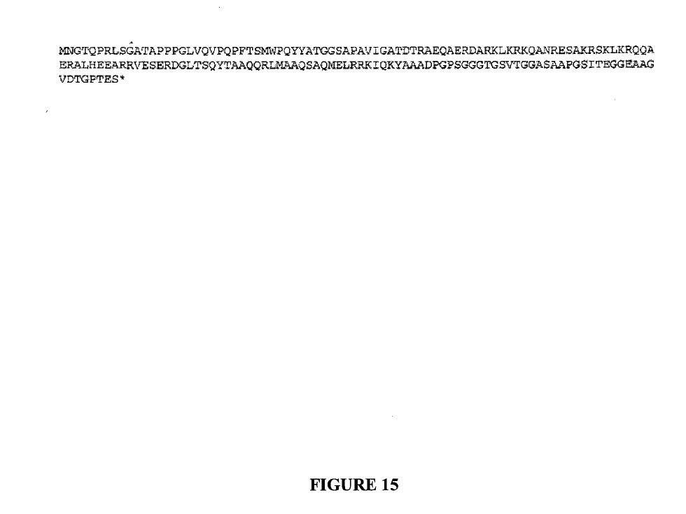

### DETAILED DESCRIPTION

The present invention is related to biosynthetic oil compositions and methods of making thereof. The present invention contemplates using plant material (i.e., for example, algae) comprising recombinant transcription inducing factors for biosynthetic oil genes. In some embodiments, the inducing factors are transcription factor regulatory proteins.

The presently contemplated invention addresses a widely recognized need for the development of biomass-based domestic production systems for high energy liquid transportation fuels. In one embodiment, the present invention contemplates inducing oil (i.e., for example, triacylglycerol) biosynthesis in microalgae. This novel inventive concept provides new insights that lay the foundation for rational engineering of algae-based production systems for high energy fuels. Initial efforts are focused on the unicellular model green alga Chlamydomonas reinhardtii with its abundance of genetic and genomic resources.

I. Oil Biosynthesis from Plant Material

Many genes encoding enzymes of storage oil biosynthesis have been isolated from plants. In particular, acyltransferases, ketoacyl-acyl carrier protein synthetases desaturases and related enzymes. Genetic engineering of these enzymes have been attempted using a single or multiple insertion of a transgene into oil crops, but a method for reliably producing a desired phenotype has not been accomplished. Present research is identifying the complexities of oil storage and membrane lipid formation, including, but not limited to, acyl group remodeling and/or the turnover of unusual fatty acids. Understanding these processes may provide a basis for the rational engineering of transgenic oil crops. In parallel with this, the domestication of plants already synthesizing useful fatty acids should be considered as a real alternative to the transgenic approach to producing novel oil crops. Murphy D. J., “Production of novel oils in plants” Curr Opin Biotechnol. 10:175-180 (1999)

Engineering oilseed crops to produce oils has been a long-standing goal of academic researchers and the biotechnology industry. Many of these oils hold great promise for use in human and animal nutritional regimes, and several others may serve as renewable chemical feedstocks that could replace petroleum-based products in industrial applications. (reviewed in Jaworski et al., “Industrial oils from transgenic plants” Curr. Opin. Plant Biol. 6:178-184 (2003); Dyer et al., “Development and potential of genetically engineered oilseeds” Seed Sci. Res. 15:255-267 (2005); and Singh et al., “Metabolic engineering of new fatty acids in plants” Curr. Opin. Plant Biol. 8:197-203 (2005). For instance, the seed oils of many exotic plant species contain high amounts of unusual fatty acids (e.g., epoxy, hydroxy, conjugated, or acetylenic) that can serve as raw materials for the production of inks, dyes, coatings, and a variety of other bio-based products. Large-scale production of these oils through traditional farming is often impossible because of the poor agronomic traits of these plant species. Furthermore, efforts to transfer genes encoding the proteins responsible for unusual fatty acid biosynthesis to higher yielding plants have generally met with limited success, with much lower amounts of the desired fatty acid accumulating in the oils of transgenic plants (15 to 30%) compared with the native plant species (up to 90%). Thelen et al., “Metabolic engineering of fatty acid biosynthesis in plants” Metab. Eng. 4:12-21 (2002). It is clear from these studies that additional genes and significantly more knowledge of seed oil biosynthesis are needed before plants can be engineered to produce industrially important oils.

It is believed that there are at least three major biosynthetic events involved in the production of seed storage oils. The first may involve the synthesis of fatty acids in plastids. The second may involve a modification of these fatty acids by enzymes located primarily in the endoplasmic reticulum (ER). The third may involve packaging of nascent fatty acids into triacylglycerols (TAGs), which subsequently accumulate in oil bodies that bud off from the ER. Research information is currently available regarding the synthesis and modification of fatty acid-containing oil body structures. (Ohlrogge et al., “Lipid Biosynthesis: Plant Cell 7:957-970. (1995); and Shanklin et al., “Desaturation and related modifications of fatty acids” Annu. Rev. Plant Physiol. Plant Mol. Biol. 49:611-641 (1998). Much less, however, is understood about the enzymes and cellular mechanisms required for the selection and transfer of fatty acids into storage TAGs.

Biochemical analyses have shown that TAG is synthesized in the ER by at least two pathways. The first involves the acyl-CoA-independent transfer of fatty acids from phospholipids to the sn-3 position of diacylglycerol to form TAG. This reaction is catalyzed by phospholipid:diacylglycerolacyltransferase (PDAT). Dahlqvist et al., “Phospholipid: diacylglycerol acyltransferase: An enzyme that catalyzes the acyl-CoA-independent formation of triacylglycerol in yeast and plants” Proc. Natl. Acad. Sci. USA 97:6487-6492 (2000); and Stahl et al., “Cloning and functional characterization of a phospholipid:diacylglycerol acyltransferase from Arabidopsis” Plant Physiol 135:1324-1335 (2004). TAG is also produced via three successive acylation reactions of the hydroxyl groups of glycerol, starting from glycerol-3-phosphate, with diacylglycerol acyltransferase (DGAT) catalyzing the committed step: the transfer of a fatty acyl moiety from acyl-CoA to the sn-3 position of diacylglycerol. (Kennedy, “Biosynthesis of complex lipids” Fed. Proc. 20:934-940 (1961). As such, it is believed that DGAT plays a role in controlling: i) the quantitative flux of fatty acids into storage TAGs (Ichihara et al., “Diacylglycerol acyltransferase in maturing safflower seeds: Its influences on the fatty acid composition of triacylglycerol and on the rate of triacylglycerol synthesis” Biochim. Biophys. Acta 958:125-129 (1988); and ii) the qualitative flux of fatty acids into storage TAGs. (Vogel et al., :Choline phosphotransferase and diacylglycerol acyltransferase (substrate specificities at a key branchpoint in seed lipid metabolism” Plant Physiol 110:923-931 (1996); and He et al., “Regulation of diacylglycerol acyltransferase in developing seeds of castor” Lipids 39:865-871. (2004).

It has been reported that a developing plant seed generates an oil storage reserve in the form of triacylglycerols. Baud et al., “An integrated overview of seed development in Arabidopsis thaliana ecotype WS” Plant Physiol. Biochem 40:151-160 (2002). The impact that glycolytic metabolic pathways have on this oil storage process has been previously studied. Glycolysis is a ubiquitous pathway thought to be essential for the production of oil in developing seeds of Arabidopsis thaliana and oil crops. Compartmentation of primary metabolism in developing embryos poses a significant challenge for testing this hypothesis and for the engineering of seed biomass production. It also raises the question whether there is a preferred route of carbon from imported photosynthate to seed oil in the embryo. Plastidic pyruvate kinase catalyzes a highly regulated, ATP-producing reaction of glycolysis. The Arabidopsis genome encodes putative isoforms of pyruvate kinases. Three genes encode subunits α, β1, and β2 of plastidic pyruvate kinase. The plastid enzyme prevalent in developing seeds likely has a subunit composition of 4α4β1, is most active at pH 8.0, and is inhibited by glucose. Disruption of the gene encoding the β1 subunit causes a reduction in plastidic pyruvate kinase activity and 60% reduction in seed oil content. The seed oil phenotype is fully restored by expression of the β1 subunit-encoding cDNA and partially by the β2 subunit-encoding cDNA. Therefore, the identified pyruvate kinase catalyzes a crucial step in the conversion of photosynthate into oil, suggesting a preferred plastid route from its substrate phosphoenolpyruvate to fatty acids. Andre et al., “A Heteromeric Plastidic Pyruvate Kinase Complex Involved In Seed Oil Biosynthesis in Arabidopsis” The Plant Cell 19:2006-2022 (2007).

### II. Biosynthetic Oil Producing Genes

Oil biosynthesis in algae has been reported to occur under stress conditions (i.e., for example, nutrient stress). The present invention contemplates using novel protein transcription factors to engineer oil biosynthesis and oil yield in algae. The present invention also contemplates novel genes targets for the engineering of oil content in microalgae.

It is generally believed that many algae species including, but not limited to, Chlamydomonas reinhardtii accumulate biosynthetic oils (i.e., for example, triacylglycerols) when cultures enter a stationary cell cycle phase subsequent to nutrient limitation. In one embodiment, the present invention contemplates methods for identifying microalgal regulatory genes encoding biosynthetic oil regulatory enzymes and/or biosynthetic oil regulatory factors.

A. Biosynthetic Oil Producing Enzymes

In one embodiment, the present invention contemplates biosynthetic oil genes encoding diacylglycerol acyltransferases (DGATs). In one embodiment, the DGAT synthesizes a biosynthetic oil. In one embodiment, the biosynthetic oil comprises a triacylglycerol.

DGAT enzyme activity are believed to be encoded by at least two classes of genes in eukaryotic cells. The type 1 class of DGAT enzymes (DGAT1) was discovered first in mouse based on homology with mammalian acyl-CoA:cholesterol acyltransferase genes. Cases et al., “Diacylglycerol acyltransferase in maturing oil seeds of maize and other species” Plant Physiol. 82:813-820 (1998). Subsequently, other DGAT1 genes were identified and characterized in several plant species. Hobbs et al., “Cloning of a cDNA encoding diacylglycerol acyltransferase from Arabidopsis thaliana and its functional expression” FEBS Lett. 452:145-149 (1999); Zou et al., “The Arabidopsis thaliana TAG1 mutant has a mutation in a diacylglycerol acyltransferase gene” Plant J. 19:645-653.1999; Bouvier-Nave' et al., “Expression in yeast and tobacco of plant cDNAs encoding acyl CoA:diacylglycerol acyltransferase” Eur. J. Biochem 267:85-96 (2000); Nykiforuk et al., “Characterization of cDNAs encoding diacylglycerol acyltransferase from cultures of Brassica napus and sucrose-mediated induction of enzyme biosynthesis” Biochim. Biophys. Acta 1580:95-109 (2002); He et al., “Cloning and characterization of a cDNA encoding diacylglycerol acyltransferase from castor bean” Lipids 39:311-318 (2004); Milcamps et al., “Isolation of a gene encoding a 1,2-diacylglycerol-sn-acetyl-CoA acetyltransferase from developing seeds of Euonymus alatus” J. Biol. Chem. 280:5370-5377 (2005).

In Arabidopsis thaliana, the DGAT1 gene has been shown to contribute significantly to TAG biosynthesis. In one study, TAG biosynthesis was induced by DGAT1 overexpression. Jako et al., “Seed-specific over-expression of an Arabidopsis cDNA encoding a diacylglycerol acyltransferase enhances seed oil content and seed weight” Plant Physiol. 126:861-874 (2001). In another study, TAG biosynthesis was studied using mutational downregulation studies. Katavic et al., “Alteration of seed fatty acid composition by an ethyl methanesulfonate-induced mutation in Arabidopsis thaliana affecting diacylglycerol acyltransferase activity” Plant Physiol. 108:399-409 (1995); and Routaboul et al., “The TAG1 locus of Arabidopsis encodes for a diacylglycerol acyltransferase” Plant Physiol. Biochem. 37:831-840 (1999).

The type 2 class of DGAT enzymes (DGAT2) also has been identified in a number of eukaryotes, including fungi, Caenorhabditis elegans, human, and Arabidopsis. Cases et al., “Diacylglycerol acyltransferase in maturing oil seeds of maize and other species” Plant Physiol. 82:813-820 (1998); and Lardizabal et al., “DGAT2 is a new diacylglycerol acyltransferase gene family: purification, cloning, and expression in insect cells of two polypeptides from Mortierella ramanniana with diacylglycerol acyltransferase activity” J. Biol. Chem. 276:38862-38869 (2001). The physiological function(s) of these DGAT2 enzymes in plants, however, has not been determined. Characterizing the subcellular properties of these enzymes would provide new insight into the underlying mechanisms of oil biosynthesis. This knowledge may be especially important for the production of seed oils containing unusual fatty acids, because these structures are generally incompatible with normal membrane lipids and the spatial separation of lipid biosynthetic enzymes in the ER may provide an efficient mechanism for channeling these unusual fatty acids into storage oils.

In particular, one study has reported a detailed analysis of DGAT1 and DGAT2 in tung tree seeds. Seeds of the tung tree (Vernicia fordii) produce large quantities of triacylglycerols (TAGs) containing 80% eleostearic acid, an unusual conjugated fatty acid. We present a comparative analysis of the genetic, functional, and cellular properties of tung type 1 and type 2 diacylglycerol acyltransferases (DGAT1 and DGAT2), two unrelated enzymes that catalyze the committed step in TAG biosynthesis. We show that both enzymes are encoded by single genes and that DGAT1 is expressed at similar levels in various organs, whereas DGAT2 is strongly induced in developing seeds at the onset of oil biosynthesis. Expression of DGAT1 and DGAT2 in yeast produced different types and proportions of TAGs containing eleostearic acid, with DGAT2 possessing an enhanced propensity for the synthesis of trieleostearin, the main component of tung oil. Both DGAT1 and DGAT2 are located in distinct, dynamic regions of the endoplasmic reticulum (ER), and surprisingly, these regions do not overlap. Furthermore, although both DGAT1 and DGAT2 contain a similar C-terminal pentapeptide ER retrieval motif, this motif alone is not sufficient for their localization to specific regions of the ER. These data suggest that DGAT1 and DGAT2 have nonredundant functions in plants and that the production of storage oils, including those containing unusual fatty acids, occurs in distinct ER subdomains. Shockey et al., “Tung Tree DGAT1 and DGAT2 Have Nonredundant Functions in Triacylglycerol Biosynthesis and Are Localized to Different Subdomains of the Endoplasmic Reticulum” The Plant Cell 18:2294-2313 (2006).

B. Microalgal Diacylglycerol Acetyltransferase

The biochemical characterization of microalgal DGATs and their role in oil biosynthesis. The newly identified genes and their respective proteins (SEQ ID NOs: 1-15) and the functional genomic information provide novel targets for future engineering approaches towards optimizing microalgal oil production strains.

### III. Transcription Factors for Biosynthetic Oil Producing Genes

Arabidopsis transcriptional factors LEAFY COTYLEDON1 (LEC1), LEAFY COTYLEDON2 (LEC2), FUSCA3 (FUS3), ABSCISIC ACID3 (ABI3), and ABSCISIC ACIDS (ABI5) have been reported to regulate multiple aspects of seed development. In an attempt to understand the developmental control of storage product accumulation, this study reported a transcript expression time course. The sequential expression of these factors during seed fill suggests differential functionality. By extending the expression periods of the two early genes LEC1 and LEC2 in transgenic seeds, it was demonstrated that the subsequent timing of FUS3, ABI3, and ABI5 transcripts depends on LEC1 and LEC2. Because a delayed onset or reduced level of FUS3 mRNA coincided with reduction of seed oil content in the transgenic seeds, the role of FUS3 in oil deposition was suspected. An ability of FUS3 to rapidly induce fatty acid biosynthetic gene expression was confirmed using transgenic Arabidopsis seedlings expressing a dexamethasone (DEX)-inducible FUS3 and Arabidopsis mesophyll protoplasts transiently expressing the FUS3 gene. A hierarchical architecture of the transcriptional network in Arabidopsis seeds was suggested in which the oil biosynthetic pathway is integrated through the master transcriptional factor FUS3. Wang et al., “Developmental control of Arabidopsis seed oil biosynthesis” Planta 226(3):773-783 (2007).

In one embodiment, the present invention contemplates a method for controlling oil biosynthesis genes by manipulating transcription factors that induce the genes. In one embodiment, the transcription factors are ectopically expressed within the algae genome, wherein the algae produces the oil in the absence of natural inducing conditions (i.e., for example, nutrient stress). Although it is not necessary to understand the mechanism of an invention, it is believed that these factors may also be used to control oil biosynthesis at will if used in combination with regulated expression systems. In one embodiment, the method further comprises a genetically engineered high-oil production algae strain (i.e., for example, a high-throughput system comprising a recombinant protein expression platform). In one embodiment, the algae strain comprises Chlamydomonas reinhardtii. In other embodiments, other algal species are used having compatibility for the recombinant constructs detailed below. In one embodiment, the present invention contemplates a plurality of inducing transcription factors (TF), including, but not limited to, TF1, TF2, TF3, TF4, and TF5.

A. Genetic Mutant Screens

The native sequences of several transcription factors are presented herein, including, but not limited to, SEQ ID NO: 3, SEQ ID NO: 6, SEQ ID NO: 9, SEQ ID NO: 12, and SEQ ID NO: 15. In one embodiment, the present invention contemplates variants of the native transcription factors, wherein a change

Genetic mutant screens will be conducted for loss of triacylglycerol accumulation under induced conditions, and for gain of triacylglycerol biosynthesis under non-induced conditions. These mutants will be biochemically and physiologically characterized and the affected genes will be identified and characterized based on a gene tagging approach.

1. Mutated Transcription Factor Genes and Proteins

The data presented herein identify a set of C. reinhardtii mutant regulatory transcription factors that modulate triacylglycerol biosynthesis and/or regulation. In one embodiment, the mutant transcription factors comprise an activity that is between 20%-50% greater than the native transcription factor. In one embodiment, the mutant transcription factors comprise an activity that is between 55%-75% greater than the native transcription factor. In one embodiment, the mutant transcription factors comprise an activity that is between 80%-100% greater than the native transcription factor. In one embodiment, the mutant transcription factors comprise an activity that is between 110%-175% greater than the native transcription factor. In one embodiment, the mutant transcription factors comprise an activity that is at least 200% greater than the native transcription factor.

In one embodiment, the present invention contemplates a mutant regulatory transcription factor comprises a single different amino acid as compared to the native transcription factor.

In one embodiment, the present invention contemplates a mutant regulatory transcription factor comprising a plurality of different amino acids as compared to the native transcription factor. In one embodiment, the mutant comprises two different amino acids. In one embodiment, the mutant comprises three different amino acids. In one embodiment, the mutant comprises four different amino acids. In one embodiment, the mutant comprises at least five different amino acids.

B. Expression Platforms

In one embodiment, the present invention contemplates a method for ectopically expressing at least one transcription factor. In one embodiment, the expression is generated from an algae culture. In one embodiment, the algae culture comprises at least one transformed algae cell, wherein the transformed cell comprises a vector comprising a promoter capable of expressing the at least one transcription factor.

In some embodiments of the present invention, a plant oil comprising TAG is produced in vivo, by providing an organism transformed with a heterologous gene encoding a DGAT transcription regulator of the present invention and growing the transgenic organism under conditions sufficient to effect production of TAGs.

In other embodiments of the present invention, a plant oil comprising TAG is produced in vivo by transforming an organism with a heterologous gene encoding an DGAT of the present invention and growing the transgenic organism under conditions sufficient to effect production of TAGs.

1. Host Organisms

Host organisms which are transformed with a heterologous gene encoding a DGAT transcription regulator of the present invention include, but are not limited to, those organisms which naturally express triacylglycerols (TAGs) and those organisms in which it is commercially feasible to grow for harvesting in large amounts of the TAG products. Such organisms include but are not limited to, oleaginous yeast and algae, and plants and animals. Examples of yeasts include oleaginous yeast, which include but are not limited to the genera Lipomyces, Candida, Rhodotorula, Rhodosporidium and Cryptococcus, which can be grown in commercial-scale fermenters. Examples of algae include, but are not limited to, Chlamydomonas. Examples of plants include preferably oil-producing plants, such as soybean, rapeseed and canola, sunflower, cotton, corn, cocoa, safflower, oil palm, coconut palm, flax, castor, and peanut. Many commercial cultivars can be transformed with heterologous genes. In cases where that is not possible, non-commercial cultivars of plants can be transformed, and the trait for expression of a DGAT transcription regulator of the present invention may be moved to commercial cultivars by various breeding techniques.

A heterologous gene encoding an DGAT transcription regulator of the present invention, including variants or mutations of DGAT transcription regulators, includes any suitable sequence of the invention as described above. Preferably, the heterologous gene is provided within an expression vector such that transformation with the vector results in expression of the polypeptide. Suitable vectors are described herein.

A transgenic organism (i.e., for example, a transgenic C. reinhardtii) is grown under conditions sufficient to effect production of TAGs. In some embodiments of the present invention, a transgenic organism is supplied with exogenous substrates of DGAT (as, for example, in a fermenter). Such substrates can comprise sugars as carbon sources for TAG synthesis, fatty acids and glycerol used directly for the production of DAG and TAG, DAG itself, and acetic acid which will both provide a general carbon source and be used for the production of acetyl-CoA and/or diacylglycerols (DAGs). When related groups are transferred to DAG, such substrates may instead or in addition be provided to the transgenic organism; exemplary related group include but are not limited to butyrate, propionate, and cinnamate. Substrates may be supplied in various forms including, but not limited to, aqueous suspensions prepared by sonication, aqueous suspensions prepared with detergents and other surfactants, dissolution of the substrate into a solvent, and dried powders of substrates. Such forms may be added to organisms or cultured cells or tissues grown in fermenters.

In yet other embodiments of the present invention, a transgenic organism (i.e., for example, a transgenic C. reinhardtii) comprises a heterologous gene encoding a DGAT transcription regulator of the present invention operably linked to an inducible promoter, and is grown either in either the presence or absence of the an inducing agent and/or inducing environmental condition (i.e., for example, nutrient stress), or is grown and then exposed to an inducing agent. In still other embodiments of the present invention, a transgenic organism comprises a heterologous gene encoding a DGAT transcription regulator of the present invention is operably linked to a promoter which is either species, cell, and/or tissue specific or developmentally specific, and is grown to the point at which the organism is developed or the developmental stage at which the developmentally-specific promoter is activated. Such promoters include, but are not limited to, seed specific promoters.

In alternative embodiments, a transgenic organism as described above is engineered to produce greater amounts of the diacylglycerol substrate. Thus, it is contemplated that a transgenic organism may include further modifications such that fatty acid synthesis is increased, and may in addition or instead include exogenous acyltransferases and/or phosphatidic acid phosphatases.

In other embodiments of the present invention, a host organism produces large amounts of a desired substrate, such as acetyl-CoA or DAG; non-limiting examples include organisms transformed with genes encoding acetyl-CoA synthetases and/or ATP citrate lyase. In some embodiments, it is contemplated that certain DAGs will result in the synthesis of novel TAGs with desirable properties. Thus, a particularly suitable host is one which produces a high proportion of such a DAG.

In other embodiments, a host organism produces low amounts of a desired substrate such as DAG. It is contemplated that in such hosts, novel TAGs produced from an exogenous DGAT are a higher proportion of the total TAGs; advantages include less expensive purification of the novel TAGs. Non-limiting exemplary hosts include those with low flux through lipid synthetic systems or with low endogenous DGAT activity (either or both DGAT1 or DGAT2). Such hosts may occur naturally or via genetic engineering techniques. Non-limiting exemplary techniques include knock-out produced by EMS and transposon tagging.

In other embodiments of the present invention, the methods for producing TAGs further comprise collecting the TAGs produced. Several methods have been reported, and include harvesting the transgenic organisms and extracting the TAGs (see, for example, Christie, W. W. (1982) Lipid Analysis. 2^{nd} Edition (Pergamon Press, Oxford); and Kates, M (1986) Techniques of Lipidology (Elsevier, Amsterdam)). Extraction procedures preferably include solvent extraction, and typically include disrupting cells, as by chopping, mincing, grinding, and/or sonicating, prior to solvent extraction. In one embodiment, lipids are extracted from the tissue according to the method of Bligh and Dyer (1959) (Can J Biochem Physiol 37: 911-917). In yet other embodiments of the present invention, the TAGs are further purified, as for example by thin layer liquid chromatography, gas-liquid chromatography, counter current chromatography or high performance liquid chromatography.

2. Vectors

The methods of the present invention contemplate the use of at least a heterologous gene encoding an DGAT transcription regulator of the present invention operably linked to a vector comprising a promoter.

Heterologous genes intended for expression in plant cells may first be assembled in expression cassettes comprising a promoter. Many methods may be used to construct expression vectors containing a heterologous gene and appropriate transcriptional and translational control elements. These methods include, but are not limited to, in vitro recombinant DNA techniques, synthetic techniques, and in vivo genetic recombination. Such techniques are widely described in the art (See for example, Sambrook. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.).

In general, these vectors comprise a nucleic acid sequence of the invention encoding an DGAT transcription regulator of the present invention (as described above) operably linked to a promoter and other regulatory sequences (for example, enhancers, polyadenylation signals, etc.) required for expression in a plant cell.

Useful promoters include, but are not limited to, constitutive promoters, tissue-, organ-, and developmentally-specific promoters, and inducible promoters. Examples of promoters include, but are not limited to: constitutive promoter 35S of cauliflower mosaic virus; a wound-inducible promoter from tomato, leucine amino peptidase (“LAP,” Chao et al. (1999) Plant Physiol 120: 979-992); a chemically-inducible promoter from tobacco, Pathogenesis-Related 1 (PR1) (induced by salicylic acid and BTH (benzothiadiazole-7-carbothioic acid S-methyl ester)); a tomato proteinase inhibitor II promoter (PIN2) or LAP promoter (both inducible with methyl jasmonate); a heat shock promoter (U.S. Pat. No. 5,187,267) (herein incorporated by reference); a tetracycline-inducible promoter (U.S. Pat. No. 5,057,422) (herein incorporated by reference); and seed-specific promoters, such as those for seed storage proteins (for example, phaseolin, napin, oleosin, and a promoter for soybean beta conglycin (Beachy et al. (1985) EMBO J. 4: 3047-3053)). All references cited herein are incorporated by reference in their entirety.

The expression cassettes may further comprise any sequences required for expression of mRNA. Such sequences include, but are not limited to, transcription terminators, enhancers such as introns, viral sequences, and sequences intended for the targeting of the gene product to specific organelles and cell compartments.

A variety of transcriptional terminators are available for use in expression of sequences using the promoters of the present invention. Transcriptional terminators are responsible for the termination of transcription beyond the transcript and its correct polyadenylation. Appropriate transcriptional terminators and those which are known to function in plants include, but are not limited to, the CaMV 35S terminator, the tm1 terminator, the pea rbcS E9 terminator, and the nopaline and octopine synthase terminator (See for example, Odell et al. (1985) Nature 313:810; Rosenberg et al. (1987) Gene, 56:125; Guerineau et al. (1991) Mol. Gen. Genet., 262:141; Proudfoot (1991) Cell, 64:671; Sanfacon Et al. Genes Dev., 5:141; Mogen et al. (1990) Plant Cell, 2:1261; Munroe et al. (1990) Gene, 91:151; Ballad et al. (1989) Nucleic Acids Res. 17:7891; Joshi et al. (1987) Nucleic Acid Res., 15:9627).

In addition, in some embodiments, constructs for expression of the gene of interest include one or more of sequences found to enhance gene expression from within the transcriptional unit. These sequences can be used in conjunction with the nucleic acid sequence of interest to increase expression in plants. Various intron sequences have been shown to enhance expression, particularly in monocotyledonous cells. For example, the introns of the maize Adh1 gene have been found to significantly enhance the expression of the wild-type gene under its cognate promoter when introduced into maize cells (Calais et al. (1987) Genes Develop. 1: 1183). Intron sequences have been routinely incorporated into plant transformation vectors, typically within the non-translated leader.

In some embodiments of the present invention, the construct for expression of the nucleic acid sequence of interest also includes a regulator such as a nuclear localization signal (Calderone et al. (1984) Cell 39:499; Lassoer et al. (1991) Plant Molecular Biology 17:229), a plant translational consensus sequence (Joshi (1987) Nucleic Acids Research 15:6643), an intron (Luehrsen and Walbot (1991) Mol. Gen. Genet. 225:81), and the like, operably linked to the nucleic acid sequence encoding a DGAT transcription regulator.

In preparing a construct comprising a nucleic acid sequence encoding DGAT transcription regulators of the present invention, various DNA fragments can be manipulated, so as to provide for the DNA sequences in the desired orientation (for example, sense or antisense) orientation and, as appropriate, in the desired reading frame. For example, adapters or linkers can be employed to join the DNA fragments or other manipulations can be used to provide for convenient restriction sites, removal of superfluous DNA, removal of restriction sites, or the like. For this purpose, in vitro mutagenesis, primer repair, restriction, annealing, resection, ligation, or the like is preferably employed, where insertions, deletions or substitutions (for example, transitions and transversions) are involved.

Numerous transformation vectors are available for plant cell transformation. The selection of a vector for use will depend upon the preferred transformation technique and the target species for transformation. For certain target species, different antibiotic or herbicide selection markers are preferred. Selection markers used routinely in transformation include the nptII gene which confers resistance to kanamycin and related antibiotics (Messing and Vierra (1982) Gene 19: 259; Bevan et al. (1983) Nature 304:184), the bar gene which confers resistance to the herbicide phosphinothricin (White et al. (1990) Nucl Acids Res. 18:1062; Spencer et al. (1990) Theor. Appl. Genet. 79:625), the hph gene which confers resistance to the antibiotic hygromycin (Blochlinger and Diggelmann (1984) Mol. Cell. Biol. 4:2929), and the dhfr gene, which confers resistance to methotrexate (Bourouis et al. (1983) EMBO J., 2:1099).

In some embodiments, the vector is adapted for use in an Agrobacterium mediated transfection process (See for example, U.S. Pat. Nos. 5,981,839; 6,051,757; 5,981,840; 5,824,877; and 4,940,838; all of which are incorporated herein by reference). Construction of recombinant plasmids encoding a DGAT transcription regulator in general follows methods typically used with the more common bacterial vectors, such as pBR322. Additional use can be made of accessory genetic elements sometimes found with the native plasmids and sometimes constructed from foreign sequences. These may include but are not limited to structural genes for antibiotic resistance as selection genes.

Exemplary systems of using recombinant plasmid vectors that are compatible with the present invention include, but are not limited to the “conintegrate” and “binary” systems. In the “cointegrate” system, the shuttle vector containing the gene of interest is inserted by genetic recombination into a non-oncogenic plasmid that contains both the cis-acting and trans-acting elements required for plant cell transformation as, for example, in the pMLJ1 shuttle vector and the non-oncogenic plasmid pGV3850. The second system is called the “binary” system in which two plasmids are used; the gene of interest is inserted into a shuttle vector containing the cis-acting elements required for plant transformation. The other necessary functions are provided in trans by the non-oncogenic plasmid as exemplified by the pBIN19 shuttle vector and the non-oncogenic plasmid PAL4404. These and other vectors useful for these systems are commercially available.

In other embodiments of the invention, the nucleic acid sequence of interest is targeted to a particular locus on the plant genome. Site-directed integration of the nucleic acid sequence of interest into the plant cell genome may be achieved by, for example, homologous recombination. Generally, plant cells are incubated with an organism comprising a targeting vector in which sequences that are homologous to a DNA sequence inside the target locus are flanked by transfer-DNA (T-DNA) sequences. U.S. Pat. No. 5,501,967 (herein incorporated by reference). Homologous recombination may be achieved using targeting vectors which contain sequences that are homologous to any part of the targeted plant gene, whether belonging to the regulatory elements of the gene, or the coding regions of the gene. Homologous recombination may be achieved at any region of a plant gene so long as the nucleic acid sequence of regions flanking the site to be targeted is known.

In yet other embodiments, the nucleic acids of the present invention are utilized to construct vectors derived from plant (+) RNA viruses (i.e., for example, brome mosaic virus, tobacco mosaic virus, alfalfa mosaic virus, cucumber mosaic virus, tomato mosaic virus, and combinations and hybrids thereof). Generally, the inserted DGAT transcription regulator polynucleotide of the present invention can be expressed from these vectors as a fusion protein (for example, coat protein fusion protein) or from its own subgenomic promoter or other promoter. Methods for the construction and use of such viruses are described. U.S. Pat. Nos. 5,846,795; 5,500,360; 5,173,410; 5,965,794; 5,977,438; and 5,866,785, all of which are incorporated herein by reference.

In some embodiments of the present invention the nucleic acid sequence of interest is introduced directly into a plant. One vector useful for direct gene transfer techniques in combination with selection by the herbicide Basta (or phosphinothricin) is a modified version of the plasmid pCIB246, with a CaMV 35S promoter in operational fusion to the E. coli GUS gene and the CaMV 35S transcriptional terminator (WO 93/07278).

3. Transformation Techniques

In one embodiment, the present invention contemplates a composition comprising a nucleic acid sequence encoding a DGAT transcription regulator of the present invention that is operatively linked to an appropriate promoter and inserted into a suitable vector for a particular transformation technique. Recombinant DNA, such as that described above, can be introduced into a plant cell in a number of ways. The choice of any specific method might depend on the type of plant targeted for transformation. In some embodiments, a vector is maintained episomally (i.e., for example, transient transformation). In other embodiments, a vector is integrated into the genome (i.e., for example, stable transformation).

In some embodiments, direct transformation in the plastid genome is used to introduce the vector into a plant cell. U.S. Pat. Nos. 5,451,513; 5,545,817; 5,545,818; PCT application WO 95/16783 (all references herein incorporated by reference). The basic technique for chloroplast transformation involves introducing regions of cloned plastid DNA flanking a selectable marker together with the nucleic acid encoding the RNA sequences of interest into a suitable target tissue (i.e., for example, using biolistics or protoplast transformation with calcium chloride or polyethylene glycol). The 1 kb to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation. (Svab et al. (1990) PNAS, 87:8526; Staub and Maliga, (1992) Plant Cell, 4:39). The presence of cloning sites between these markers allowed creation of a plastid targeting vector introduction of foreign DNA molecules (Staub and Maliga (1993) EMBO J., 12:601). Substantial increases in transformation frequency may be obtained by replacement of the recessive rRNA or r-protein antibiotic resistance genes with a dominant selectable marker, such as a bacterial aadA gene encoding the spectinomycin-detoxifying enzyme aminoglycoside-3′-adenyltransferase (Svab and Maliga (1993) PNAS, 90:913). Other selectable markers have been shown useful for plastid transformation. Plants homoplasmic for plastid genomes containing the two nucleic acid sequences separated by a promoter of the present invention are obtained, and are preferentially capable of high expression of the RNAs encoded by the DNA molecule.

In other embodiments, vectors useful in the practice of the present invention are microinjected directly into plant cells by use of micropipettes to mechanically transfer the recombinant DNA (Crossway (1985) Mol. Gen. Genet, 202:179). In still other embodiments, the vector is transferred into the plant cell by using polyethylene glycol (Krens et al. (1982) Nature, 296:72; Crossway et al. (1986) BioTechniques, 4:320); fusion of protoplasts with other entities, either minicells, cells, lysosomes or other fusible lipid-surfaced bodies (Fraley et al. (1982) Proc. Natl. Acad. Sci., USA, 79:1859); protoplast transformation (EP 0 292 435); direct gene transfer (Paszkowski et al. (1984) EMBO J., 3:2717; Hayashimoto et al. (1990) Plant Physiol. 93:857).

In still further embodiments, the vector may also be introduced into the plant cells by electroporation (Fromm, et al. (1985) Proc. Natl. Acad. Sci. USA 82:5824; Riggs et al. (1986) Proc. Natl. Acad. Sci. USA 83:5602). In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus.

In yet other embodiments, the vector is introduced through ballistic particle acceleration using devices (for example, available from Agracetus, Inc., Madison, Wis. and Dupont, Inc., Wilmington, Del.). (See for example, U.S. Pat. No. 4,945,050 (herein incorporated by reference); and McCabe et al. (1988) Biotechnology 6:923). See also, Weissinger et al. (1988) Annual Rev. Genet. 22:421; Sanford et al. (1987) Particulate Science and Technology, 5:27 (onion); Svab et al. (1990) Proc. Natl. Acad. Sci. USA, 87:8526 (tobacco chloroplast); Christou et al. (1988) Plant Physiol., 87:671 (soybean); McCabe et al. (1988) Bio/Technology 6:923 (soybean); Klein et al. (1988) Proc. Natl. Acad. Sci. USA, 85:4305 (maize); Klein et al. (1988) Bio/Technology, 6:559 (maize); Klein et al. (1988) Plant Physiol., 91:4404 (maize); Fromm et al. (1990) Bio/Technology, 8:833; and Gordon-Kamm et al. (1990) Plant Cell, 2:603 (maize); Koziel et al. (1993) Biotechnology, 11:194 (maize); Hill et al. (1995) Euphytica, 85:119 and Koziel et al. (1996) Annals of the New York Academy of Sciences 792:164; Shimamoto et al. (1989) Nature 338: 274 (rice); Christou et al. (1991) Biotechnology, 9:957 (rice); Datta et al. (1990) Bio/Technology 8:736 (rice); European Patent Application EP 0 332 581 (orchardgrass and other Pooideae); Vasil et al. (1993) Biotechnology, 11: 1553 (wheat); Weeks et al. (1993) Plant Physiol., 102: 1077 (wheat); Wan et al. (1994) Plant Physiol. 104: 37 (barley); Jaime et al. (1994) Theor. Appl. Genet. 89:525 (barley); Knudsen and Muller (1991) Planta, 185:330 (barley); Umbeck et al. (1987) Bio/Technology 5: 263 (cotton); Casas et al. (1993) Proc. Natl. Acad. Sci. USA 90:11212 (sorghum); Somers et al. (1992) Bio/Technology 10:1589 (oat); Torbert et al. (1995) Plant Cell Reports, 14:635 (oat); Weeks et al. (1993) Plant Physiol., 102:1077 (wheat); Chang et al., WO 94/13822 (wheat) and Nehra et al. (1994) The Plant Journal, 5:285 (wheat).

In addition to direct transformation, in some embodiments, the vectors comprising a nucleic acid sequence encoding a DGAT transcription regulator of the present invention are transferred using Agrobacterium-mediated transformation (Hinchee et al. (1988) Biotechnology, 6:915; Ishida et al. (1996) Nature Biotechnology 14:745). Agrobacterium is a representative genus of the gram-negative family Rhizobiaceae. Its species are responsible for plant tumors such as crown gall and hairy root disease. In the dedifferentiated tissue characteristic of the tumors, amino acid derivatives known as opines are produced and catabolized. The bacterial genes responsible for expression of opines are a convenient source of control elements for chimeric expression cassettes. Heterologous genetic sequences (i.e., for example, nucleic acid sequences operatively linked to a promoter of the present invention), can be introduced into appropriate plant cells, by means of the Ti plasmid of Agrobacterium tumefaciens. The Ti plasmid is transmitted to plant cells on infection by Agrobacterium tumefaciens, and is stably integrated into the plant genome (Schell (1987) Science, 237: 1176). Species that are susceptible infection by Agrobacterium may be transformed in vitro. Alternatively, plants may be transformed in vivo, such as by transformation of a whole plant by Agrobacteria infiltration of adult plants, as in a “floral dip” method (Bechtold N, Ellis J, Pelletier G (1993) Cr. Acad. Sci. III-Vie 316: 1194-1199).

C. Differential Expression of Biosynthetic Oil Producing Genes

The data presented herein identify a set of differentially expressed genes comprising regulons and regulators for microalgal triacylglycerol biosynthesis. In one embodiment, the differentially expressed genes are identified under induced conditions. In one embodiment, the differentially expressed genes are identified under non-induced conditions. Global expression analysis is but one method that is capable of determining possible set of differentially expressed genes of the transcription factor in question. Other methods, of course, are also useful.

The degree of differentiation or physiological state of a cell, a tissue or an organism is characterized by a specific expression status, i.e., the degree of transcriptional activation of all genes or particular groups of genes. The molecular basis for numerous biological processes that result in a change in this state is the coordinated transcriptional activation or inactivation of particular genes or groups of genes in a cell, an organ or an organism. Characterization of this expression status is indicative to answering many biological questions. Changes in gene expression in response to a stimulus, a developmental stage, a pathological state or a physiological state are important in determining the nature and mechanism of the change and in finding cures that could reverse a pathological condition. Patterns of gene expression are also expected to be useful in the diagnosis of pathological conditions, and for example, may provide a basis for the sub-classification of functionally different subtypes of cancerous conditions.

1. Traditional Differential Expression Analysis Techniques

Several methods that can analyze the expression status of genes are presently used. For example, differential display RT-PCR (DDRT) is one method for analyzing differential gene expression in which subpopulations of complementary DNA (cDNA) are generated by reverse transcription of mRNA by using a cDNA primer with a 3′ extension (i.e., for example, by using two bases). Random 10-base primers are then used to generate PCR products of transcript-specific lengths. If the number of primer combinations used is large enough, it is statistically possible to detect almost all transcripts present in any given sample. PCR products obtained from two or more samples are then electrophoresed next to one another on a gel and differences in expression are directly compared. Differentially expressed bands can be cut out of the gel, reamplified and cloned for further analysis.

In one embodiment of DDRT it is possible to enrich the PCR amplification products for a particular subgroup of all mRNA molecules, e.g., members of a particular gene family by using one primer which has a sequence specific for a gene family in combination with one of the 10 base random primers. Liang et al., Science, 257:967-971 (1992); Liang et al., Nucleic Acids Res 21:3269-3275 (1993); Bauer et al., Nucleic Acids Res., 21:4272-4280 (1993); Stone et al., Nucleic Acids Res., 22:2612-2618 (1994); Wang et al., Biotechniques 18:448-453 (1995); WO 93/18176; and DE 43 17 414 (all references herein incorporated by reference in their entirety).

There are a number of disadvantages to the experimental design of DDRT. The differential banding patterns are often only poorly reproducible. Due to the design of the primers even the use of longer random primers of, e.g., 20 bases in length does not satisfactorily solve the problem of reproducibility. Ito et al., FEBS Lett 351:231-236 (1994). In order to evaluate a significant portion of differentially expressed genes, a large number of primer combinations must be used and multiple replicates of each study must be done. The method often results in a high proportion of false positive results and rare transcripts cannot be detected in many DDRT studies. Bertioli et al., Nucleic Acids Res. 23:4520-4523 (1995.)

Due to the non-stringent PCR conditions and the use of only one arbitrary primer further analysis by sequencing is necessary to identify the gene. Sequencing of selected bands is problematic since the same primer often flanks DDRT products at both ends so that direct sequencing is not possible and an additional cloning step is necessary. Due to the use of short primers, a further reamplification step with primer molecules extended on the 5′ side is necessary even if two different primers flank the product. Finally, due to the use of random primers, it is never quite possible to be sure that the primer combinations recognize all transcripts of a cell. This applies, even when using a high number of primers, to studies which are intended to detect the entirety of all transcripts as well as to studies which are directed towards the analysis of a subpopulation of transcripts such as a gene family.

A variant of DDRT, known as GeneCalling, has recently been described which addresses some of these problems. Shimkets et al., Nat Biotechnol. 17:798-803 (1999). In this method, multiple pairs of restriction endonucleases are used to prepare specific fragments of a cDNA population prior to amplification with pairs of universal primers. This improves the reproducibility of the measurements and the false positive rate, but the patterns are very complex and identification of individual transcripts requires the synthesis of a unique oligonucleotide for each gene to be tested. In addition, the quantitative data obtained are apparently significant only for changes above 4-fold and only a weak correlation with other techniques is obtained. The ability of the technique to distinguish the gene-specific band from the complex background for any arbitrarily chosen gene has not been documented.

AFLP based mRNA fingerprinting further addresses some of the deficiencies of DDRT. AFLP allows for the systematic comparison of the differential expression of genes between RNA samples. Habu et al, Biochem Biophys Res Commun 234:516-21 (1997) The technique involves the endonuclease digestion of immobilized cDNA by a single restriction enzyme. The digested fragments are then ligated with a linker specific for the restriction cut site. The tailed fragments are subsequently amplified by PCR employing primers complementary to the linkers added to the digest with the addition of variable nucleotides at the 3′ end of the primers. The products of the amplification are visualized by PAGE and banding patterns compared to reveal differences in RNA transcription patterns between samples. Although AFLP based RNA fingerprinting provides a indication of the RNA message present in a given sample, it fails to restrict the potential number of signals produced by each individual RNA strand. With this technique, each RNA strand may potentially produce multiple fragments and therefore multiple signals upon amplification. This failure to restrict the number of signals from each message complicates the results that must be evaluated.

Methods have been described for examining the expression of homologous genes in plant polyploids in which the techniques of RT-PCR. and restriction fragment length polymorphism (RFLP) analysis are combined with one another. Song et al., Plant Mol Biol. 26:1065-1071 (1994). This method uses a cDNA produced from RNA by reverse transcription, and then amplified by using two gene-specific primers. The amplification products are transcript-specifically shortened by endonuclease cleavage, separated by electrophoresis according to their length, cloned, and then analyzed by sequencing. This method has the disadvantage of low sensitivity, as a cloning step is necessary to characterize the expression products. A further disadvantage of this method is that gene specific sequence information must be available on at least two regions within the analyzed genes in order to design suitable primers.

In principle, gene expression data for a particular biological sample could be obtained by large-scale sequencing of a cDNA library. The role of sequencing cDNA, generated by reverse transcription from mRNA, has been debated for its value in the human genome project. Proponents of genomic sequencing have argued the difficulty of finding every mRNA expressed in all tissues, cell types, and developmental stages. It is also believed that cDNA libraries do not provide all sequences corresponding to structural and regulatory polypeptides. Putney et al., Nature, 302:718-21 1983. In addition, libraries of cDNA may to be dominated by repetitive elements, mitochondrial genes, ribosomal RNA genes, and other nuclear genes comprising common or housekeeping sequences. While some mRNAs are abundant, others are rare, resulting in cellular quantities of mRNA from various genes that can vary by several orders of magnitude. Therefore, sequencing of transcribed regions of the genome using cDNA libraries has been considered unsatisfactory.

Techniques based on cDNA subtraction or differential display can be used to compare gene expression patterns between two cell types. Hedrick et al., Nature 308:153-8 (1984); and Liang et al., Science 257:967-971 (1992). These techniques, however, provide only a partial analysis, with no quantitative information regarding the abundance of messenger RNA. Expressed sequence tags (EST) have been valuable for gene discovery. (Adams et al., Nat Genet, 4:373-4380 (1993); and Okubo et al., Nat Genet. 2:173-179 (1992), but like Northern blotting, RNase protection, and reverse transcriptase-polymerase chain reaction (RT-PCR) analysis, this approach only evaluates a limited number of genes at a time.

2. Global Gene Expression

Several strategies for global gene expression analysis have recently become available. For example, Serial Analysis of Gene Expression (SAGE) is based on the use of short (i.e., for example, 9-10 base pairs) nucleotide sequence tags that identify a defined position in an mRNA and are used to ascertain the identity of the corresponding transcript and gene. U.S. Pat. No. 5,866,330 To Kinzler et al., (1995)(herein incorporated by reference). The cDNA tags are generated from mRNA samples, randomly paired, concatenated, cloned, and sequenced. While this method allows the analysis of a large number of transcripts, the identification of individual genes requires sequencing of tens of thousands of tags for comparison of even a small number of samples. Although SAGE provides a comprehensive picture of gene expression, it is difficult to specifically direct the analysis at a small subset of the transcriptome. (Zhang et al., Science 276:1268-1272 (1997); and Velculescu et al., Cell 88:243-251 (1995). Data on the most abundant transcripts is the easiest and fastest to obtain, while about a megabase of sequencing data is needed for confident analysis of low abundance transcripts.

Another global expression analysis method utilizes hybridization of cDNAs or mRNAs to microarrays containing hundreds or thousands of individual cDNA fragments or oligonucleotides specific for particular genes or ESTs. The matrix for hybridization is either a DNA chip, a slide or a membrane. This method can be used to direct a search towards specific subsets of genes, but cannot be used to identify novel genes as are expensive to produce. DeRisi et al., Nature Genetics, 14:457-460 (1996); and Schena et al., Science 270:467-470 (1995). For those methods using cDNA arrays, a library of individually cloned DNA fragments must be maintained with at least one clone for each gene to be analyzed. Because much of the expense of utilizing microarrays lies in maintaining the fragment libraries and programming equipment to construct the microarray, it is only cost-efficient to produce large numbers of identical arrays. These two techniques lack the flexibility to easily change the subset of the transcriptome being analyzed or to focus on smaller subsets of genes for more detailed analyses.

As described above, current techniques for analysis of gene expression either monitor one gene at a time, are designed for the simultaneous and therefore more laborious analysis of thousands of genes or do not adequately restrict the signal to message ratio. There is a need for improved methods which encompass both rapid, detailed analysis of global expression patterns of genes as well as expression patterns of defined sets of genes for the investigation of a variety of biological applications. This is particularly true for establishing changes in the pattern of gene expression in the same cell type, for example, in different developmental stages, under different physiologic or pathologic conditions, when treated with different pharmaceuticals, mutagens, carcinogens, etc. Identification of differential patterns of expression has several utilities, including the identification of appropriate therapeutic targets, candidate genes for gene therapy (including gene replacement), tissue typing, forensic identification, mapping locations of disease-associated genes, and for the identification of diagnostic and prognostic indicator genes.

D. High-Throughput cDNA Pyrosequencing

A high-throughput cDNA pyrosequencing experiment will be conducted under induced and non-induced conditions to generate a deep set of expressed sequence tags for comparative transcriptional profiling.

Pyrosequencing is an iterative technique whereby only one of the four deoxynucleotide triphosphates (“dNTPs”) is present in each of the iterative assays to enable each deoxynucleotide triphosphate (“dNTP”) to be tested at each position of the sequence. Thus all of the components necessary for DNA synthesis are never present simultaneously.

For example, pyrosequencing may be carried out as follows: Twenty-five μl of biotinylated PCR product was immobilized onto streptavidin-coated paramagnetic beads (Dynal AS, Oslo, Norway) using Binding-Washing buffer (5 mM Tris-HCl, 1M NaCl, 0.5 mM EDTA, 0.05% Tween 20, pH 7.6) in a total volume of 90 μl at 43° C. for 30 mm. Single-stranded (ss) DNA was obtained by incubating the immobilized PCR product in 50 μl of 0.5 M NaOH for 1 mm and washing the beads once in 100μ of Binding-Washing buffer. Fifteen pmoles of detection primer KitSeq TAATTACNTGGTCAAAGGAAAC-3′, N=inosine (SEQ ID NO: 16), designed with its 3′ end immediately upstream of the splice mutation, may be allowed to hybridize onto ssDNA in 40 μl of Annealing buffer (20 mM Tris-Acetate, 5 mM MgAc.sub.2, pH 7.6) at 80° C. for 2 mm with subsequent cooling down to room temperature. Pyrosequencing was carried out using the SNP Reagent Kit containing dATPαS, dCTP, dGTP, dTTP, enzyme mixture (DNA polymerase, ATP sulfurylase, luciferase and apyrase) and substrate mixture (APS and luciferin) and the PSQ96 instrument (Pyrosequencing AB, Uppsala, Sweden). The result of the pyrosequencing assay was expressed as the ratio between the signals from the incorporated dATPaS and dGTP, standardized with the ratio of the next incorporated dATPaS and dGTP in the sequence.

### IV. Nucleic Acid and Protein Detection

A. Detection of RNA

mRNA expression may be measured by any suitable method, including but not limited to, those disclosed below.

In some embodiments, RNA is detection by Northern blot analysis. Northern blot analysis involves the separation of RNA and hybridization of a complementary labeled probe.

In other embodiments, RNA expression is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific nucleic acid (e.g., RNA) sequences by using structure-specific enzymes to cleave a complex formed by the hybridization of overlapping oligonucleotide probes.

In still further embodiments, RNA (or corresponding cDNA) is detected by hybridization to a oligonucleotide probe. A variety of hybridization assays using a variety of technologies for hybridization and detection are available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

B. Detection of Protein

In other embodiments, gene expression may be detected by measuring the expression of a protein or polypeptide. Protein expression may be detected by any suitable method. In some embodiments, proteins are detected by immunohistochemistry. In other embodiments, proteins are detected by their binding to an antibody raised against the protein. The generation of antibodies is described below.

Antibody binding may be detected by many different techniques including, but not limited to, (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to cancer markers is utilized.

In other embodiments, the immunoassay described in U.S. Pat. Nos. 5,599,677 and 5,672,480; each of which is herein incorporated by reference.

C. Remote Detection Systems

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, wherein the information is provided to medical personal and/or subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

D. Detection Kits

In other embodiments, the present invention provides kits for the detection and characterization of proteins and/or nucleic acids. In some embodiments, the kits contain antibodies specific for a protein expressed from a gene of interest, in addition to detection reagents and buffers. In other embodiments, the kits contain reagents specific for the detection of mRNA or cDNA (e.g., oligonucleotide probes or primers). In preferred embodiments, the kits contain all of the components necessary to perform a detection assay, including all controls, directions for performing assays, and any necessary software for analysis and presentation of results.

We claim:

1. A composition comprising a nucleic acid sequence encoding a biosynthetic oil gene transcription regulator.

2. The composition of claim 1, wherein said nucleic acid sequence comprises SEQ ID NO:2 (TF1).

3. The composition of claim 1, wherein said nucleic acid sequence comprises SEQ ID NO:5 (TF2).

4. The composition of claim 1, wherein said nucleic acid sequence comprises SEQ ID NO:8 (TF3).

5. The composition of claim 1, wherein said nucleic acid sequence comprises SEQ ID NO:11 (TF4).

6. The composition of claim 1, wherein said nucleic acid sequence comprises SEQ ID NO:14 (TF5).

7. The composition of claim 1, the nucleic acid sequence is derived from a plant algae.

8. The composition of claim 7, wherein said algae comprises Chlamydomonas reinhardtii.

9. A composition comprising an amino acid sequence comprising a biosynthetic oil gene transcription regulator.

10. The composition of claim 9, wherein said amino acid sequence comprises SEQ ID NO:3 (TF1).

11. The composition of claim 9, wherein said amino acid sequence comprises SEQ ID NO:6 (TF2).

12. The composition of claim 9, wherein said amino acid sequence comprises SEQ ID NO:9 (TF3).

13. The composition of claim 9, wherein said amino acid sequence comprises SEQ ID NO:12 (TF4).

14. The composition of claim 9, wherein said amino acid sequence comprises SEQ ID NO:15 (TF5).

15. The composition of claim 9, wherein said amino acid sequence is derived from a plant algae.

16. The composition of claim 15, wherein said algae comprises Chlamydomonas reinhardtii.

17. A method comprising:
a) providing;
i) a nucleic acid sequence comprising a biosynthetic oil gene transcription regulator;
ii) at least one plant cell comprising a biosynthetic oil gene; and
iii) a vector comprising a promoter capable of expressing said transcription regulator;
b) ligating said nucleic acid sequence to said vector, wherein said nucleic acid sequence is operably linked to said vector; and
c) transfecting said algae cell with said operably linked vector such that said nucleic acid sequence expresses said transcription regulator.

18. The method of claim 17, wherein said method further comprises step (d) inducing said biosynthetic oil gene with said transcription regulator, wherein said biosynthetic oil gene expression is upregulated.

19. The method of claim 17, wherein said gene expression is ectopic.

20. The method of claim 18, wherein said biosynthetic oil gene is upregulated between 1.5-3 fold.

21. The method of claim 18, wherein said biosynthetic oil gene is upregulated between 3.5-5 fold.

22. The method of claim 18, wherein said biosynthetic oil gene is upregulated between 5.5-7 fold.

23. The method of claim 18, wherein said biosynthetic oil gene is upregulated between 7.5-10 fold.

24. The method of claim 17, wherein said biosynthetic oil gene encodes a diacylglycerol acyltransferase enzyme.

25. The method of claim 24, wherein said enzyme produces a fatty acid.

26. The method of claim 25, wherein said fatty acid comprises triacylglycerol.

27. The method of claim 25, wherein said method further comprises step (e) collecting the fatty acid, thereby forming a biosynthetic oil.

28. The method of claim 17, wherein said plant cell comprises an algae cell.

29. The method of claim 28, wherein said algae cell comprises a Chlamydomonas reinhardtii cell.

30. A transgenic plant cell line, wherein said line comprises a nucleic acid sequence encoding a heterologous DGAT transcription regulator.

31. The cell line of claim 30, wherein said nucleic acid sequence comprises SEQ ID NO:2 (TF1).

32. The cell line of claim 30, wherein said nucleic acid sequence comprises SEQ ID NO:5 (TF2).

33. The cell line of claim 30, wherein said nucleic acid sequence comprises SEQ ID NO:8 (TF3).

34. The cell line of claim 30, wherein said nucleic acid sequence comprises SEQ ID NO:11 (TF4).

35. The cell line of claim 30, wherein said nucleic acid sequence comprises SEQ ID NO:14 (TF5).

36. The cell line of claim 30, wherein said plant cell comprises an algae species.

37. The cell line of claim 36, wherein said algae species comprises Chlamydomonas reinhardtii.


SEQUENCE LISTING (16 sequences)

SEQ ID NO: 1 - DNA, 3581 bp, Chlamydomonas reinhardtii
cgacatgttcgcgaactggctccacagcgtactttgttgagttttgttgcacgtaccactgacattgcgataacatataaggctagagatcaagttaaagcaggggcgcgagtcggccgacggccccgttgcatggggtgcttgctggacggtgaagacgagagctctcgcctcaaaggttcagtccccaacgcttgcacatttgccctaaacgcaatactccccaacaacacaaatataccacacctctatacgcgagaatgtcgagttgcgtcgtgtgcgcggccgcagcggtcgtttggtgccagaatgacaaggcgctgctttgcaaggactgcgatgtgcgcatccacaccagcaacgcggtgaggagtgcccccaagtataggagatgtgccccacttataggaggatcaggccaggtgccctcgctggcagggccgagcttgaagctgccgcttgctgcagaagttgctgacaaggttgtttgcgcctttgcaggtcgctgcgcgccatacccgcttcgtgccctgccagggctgcaacaaggccggtgctgcgctctactgcaagtgcgacgccgcgcacatgtgcgaggcttgccacagctccaaccccctagctgctacgcacgagaccgagccggtggcgccgctgccgtcagtcgagcaggtgcgaaaaaagaagctgacgatggtggcgtcatctttataaccggacttctcgctctcttttacagggcgctgcaccggagcctcaggtcctgaacatgccctgcgagtctgtggcgcagtctgcggccagccccgcggcttggtttgtggacgacgagaagatgggcacgaccagcttctttgatgcgcctgcggtgctgtcgccctcgggcagcgaggccgtggtgcccgtcatgtccgcccctatcgaggacgagtttgcattcgcggccgccccggcgacgttcaaggaaatcaaggacaagctcgagttcgaggtgcgtttcctgctcccacatgtcgcgggcgctgcattcccttacgcatgcattggatagaagcttacgtattgccccgtttttgcctttcctgaccgccaggctctggacctggacaacaactggctcgacatgggcttcgatttcactgatatcctgtccgacggcccctctgatggtaagggcacacatcacacagcttctctttcacggcctgcggagggccgatgggtacgatgaacccggggtttccaatctgggttgcgcgatcgcctgactccactatggctctctgccctcctctcgtgcagtgggcctggtccccaccttcgatgccgtcgatgaggccgcggatgccgtggctgacgctatcgtgcccaccttcgaggaggagcagccccagttacagcagcaggagcccctggtgctggctcccgccccggaggagtcggctgctagccgcaagcgcgctgccgccgaggaggccgcggaggagccggccgccaaggtgccggccctgactcaccaggcgctgctgcaggcgcaggccgccgccttccaggccgtgccccaggcgtcagcgctgttcttccagccgcagatgctggccgcgctgccgcacctgccgctgctgcagcagcccatgatgccggcagccgtcgccccggcgcccgtgcccaagagcggcagcgccgccgccagcgcggccctcgccgccggtgccaacctgactcgcgagcagcgcgtggcgcgctaccgcgagaagcggaagaaccgctctttcgccaagaccatccgctacgcttcccgcaaggcgtatgcggagatccgcccccgcattaagggccgcttcgccaagaaggaggagattgaggcctggaaggcggcgcacggcggcgacgacgccattgttcccgaggtcctggacgctgagtgctaaggaagctgactaacctcgcgtcgggtgtgcgcgcggaccggatttgaggagtggcatgactacgtggttacgtgcatggggatgcattgggggatagtgtgctgcgctgcgggcgaggggcgcacagcggtgtgatgggagccgatattgccctgtcggggtgagggcgccagtcagccactcacagggctaagccgctgggtgcagctgctactttcatcgatctcccttttcaagtcttaatatggttgttaattgttttgttgaagacgcgatttagttcaaggcctataagcgcccctgtgcaggcgatttcatattttcggcggccggcgggcgctggtgcgcgtctgcggtgcccccgccactgcgggaatagccaggcagtcagtcctgggcctctgtggctattcggactggccaagaaatcttagctggcttggggcgcgaacatgtagcgcggtgttttcgcttgatggcgtcgaatacgagagttcaagttatagctggtcccgtgcgccgggcgcacggcgatgtaccattggctttgggctgcatgcgcgccgaccgcgatcttgtggatcatggttgtgcggttttgttcgcggaggggtgtggaccttttggccgcctgggggccgaggaatgtcaaattctgcgcagctctgtcttgagcgaaggctgctggaagtactggatgtgcgaggtgtgagttttttaagtgtgggtatagctgagcgattttaactcggcgcagggcaacggctccgcgctgctgacaggagccttgaatcggcatctgatcattgaacaccaacggcaaggcgcatcggccgctgcgctgcataaccgtgtagggcagggattaccacgtgtttgatttatcagatcctgaggaagggaggggcatgtatgccgcatggcacggcgctgtgcaggtaggcggcgaatgcgttgtgtgtcactgggattgcctgtacagccgcgaactgtgtcggcgggatcactgtggcgtcaacgcattgtgtgcggtgtgaaggccattggagcggatggccagtcggtgcagaatggcaggctgcacagttttcagacgaccccgtatcagagcctgatcggacatggcggttgctggggctcgagagagaacaaagcctgcccccgtggcacggcacgagagaagagtgagaatcgcgagagtgagtgaatcatgagcttggtgcttacgcgttgctcaggggcaccagctggcgatgctggtggggagaagacaacacgattacacgttcgatgtggttgatgggtgatgtggttgacacggattggttcatggatgttgctttgttcgcagcatgtattggatgcccgccatcattcgaggcgcccgatacaacacttggcgtccacagttcacaaggatgatggccaacactttaatgatctagttggtcgagtgacgagtcgctctttctctgctggccccacgccagaatctaaccagacgtcctggctggttcccggtggtgagacagagtgcgacatctcaagaagggacaatccgagcaaacgaaggcgcaccgtatgccgttgcagcgcggtgctgccttgactgtctcattgccctggttccgcttccctgccgccccgcca

SEQ ID NO: 2 - DNA, 2743 bp, Chlamydomonas reinhardtii
atttgccctaaacgcaatactccccaacaacacaaatataccacacctctatacgcgagaatgtcgagttgcgtcgtgtgcgcggccgcagcggtcgtttggtgccagaatgacaaggcgctgctttgcaaggactgcgatgtgcgcatccacaccagcaacgcggtcgctgcgcgccatacccgcttcgtgccctgccagggctgcaacaaggccggtgctgcgctctactgcaagtgcgacgccgcgcacatgtgcgaggcttgccacagctccaaccccctagctgctacgcacgagaccgagccggtggcgccgctgccgtcagtcgagcagggcgctgcaccggagcctcaggtcctgaacatgccctgcgagtctgtggcgcagtctgcggccagccccgcggcttggtttgtggacgacgagaagatgggcacgaccagcttctttgatgcgcctgcggtgctgtcgccctcgggcagcgaggccgtggtgcccgtcatgtccgcccctatcgaggacgagtttgcattcgcggccgccccggcgacgttcaaggaaatcaaggacaagctcgagttcgaggctctggacctggacaacaactggctcgacatgggcttcgatttcactgatatcctgtccgacggcccctctgatgtgggcctggtccccaccttcgatgccgtcgatgaggccgcggatgccgtggctgacgctatcgtgcccaccttcgaggaggagcagccccagttacagcagcaggagcccctggtgctggctcccgccccggaggagtcggctgctagccgcaagcgcgctgccgccgaggaggccgcggaggagccggccgccaaggtgccggccctgactcaccaggcgctgctgcaggcgcaggccgccgccttccaggccgtgccccaggcgtcagcgctgttcttccagccgcagatgctggccgcgctgccgcacctgccgctgctgcagcagcccatgatgccggcagccgtcgccccggcgcccgtgcccaagagcggcagcgccgccgccagcgcggccctcgccgccggtgccaacctgactcgcgagcagcgcgtggcgcgctaccgcgagaagcggaagaaccgctctttcgccaagaccatccgctacgcttcccgcaaggcgtatgcggagatccgcccccgcattaagggccgcttcgccaagaaggaggagattgaggcctggaaggcggcgcacggcggcgacgacgccattgttcccgaggtcctggacgctgagtgctaaggaagctgactaacctcgcgtcgggtgtgcgcgcggaccggatttgaggagtggcatgactacgtggttacgtgcatggggatgcattgggggatagtgtgctgcgctgcgggcgaggggcgcacagcggtgtgatgggagccgatattgccctgtcggggtgagggcgccagtcagccactcacagggctaagccgctgggtgcagctgctactttcatcgatctcccttttcaagtcttaatatggttgttaattgttttgttgaagacgcgatttagttcaaggcctataagcgcccctgtgcaggcgatttcatattttcggcggccggcgggcgctggtgcgcgtctgcggtgcccccgccactgcgggaatagccaggcagtcagtcctgggcctctgtggctattcggactggccaagaaatcttagctggcttggggcgcgaacatgtagcgcggtgttttcgcttgatggcgtcgaatacgagagttcaagttatagctggtcccgtgcgccgggcgcacggcgatgtaccattggctttgggctgcatgcgcgccgaccgcgatcttgtggatcatggttgtgcggttttgttcgcggaggggtgtggaccttttggccgcctgggggccgaggaatgtcaaattctgcgcagctctgtcttgagcgaaggctgctggaagtactggatgtgcgaggtgtgagttttttaagtgtgggtatagctgagcgattttaactcggcgcagggcaacggctccgcgctgctgacaggagccttgaatcggcatctgatcattgaacaccaacggcaaggcgcatcggccgctgcgctgcataaccgtgtagggcagggattaccacgtgtttgatttatcagatcctgaggaagggaggggcatgtatgccgcatggcacggcgctgtgcaggtaggcggcgaatgcgttgtgtgtcactgggattgcctgtacagccgcgaactgtgtcggcgggatcactgtggcgtcaacgcattgtgtgcggtgtgaaggccattggagcggatggccagtcggtgcagaatggcaggctgcacagttttcagacgaccccgtatcagagcctgatcggacatggcggttgctggggctcgagagagaacaaagcctgcccccgtggcacggcacgagagaagagtgagaatcgcgagagtgagtgaatcatgagcttggtgcttacgcgttgctcaggggcaccagctggcgatgctggtggggagaagacaacacgattacacgttcgatgtggttgatgggtgatgtggttgacacggattggttcatggatgttgctttgttcgcagcatgtattggatgcccgccatcattcgaggcgcccgatacaacacttggcgtccacagttcacaaggatgatggccaacactttaatgatctagttggt

SEQ ID NO: 3 - protein, 410 aa, Chlamydomonas reinhardtii
MSSCVVCAAAAVVWCQNDKALLCKDCDVRIHTSNAVAARHTRFVPCQGCNKAGAALYCKCDAAHMCEACHSSNPLAATHETEPVAPLPSVEQGAAPEPQVLNMPCESVAQSAASPAAWFVDDEKMGTTSFFDAPAVLSPSGSEAVVPVMSAPIEDEFAFAAAPATFKEIKDKLEFEALDLDNNWLDMGFDFTDILSDGPSDVGLVPTFDAVDEAADAVADAIVPTFEEEQPQLQQQEPLVLAPAPEESAASRKRAAAEEAAEEPAAKVPALTHQALLQAQAAAFQAVPQASALFFQPQMLAALPHLPLLQQPMMPAAVAPAPVPKSGSAAASAALAAGANLTREQRVARYREKRKNRSFAKTIRYASRKAYAEIRPRIKGRFAKKEEIEAWKAAHGGDDAIVPEVLDAEC

SEQ ID NO: 4 - DNA, 2427 bp, Chlamydomonas reinhardtii
actctgcgaaggttgctgtcgttgattttgcgcagtcaatcagttggatgtcagagcgtttgtatcggtgtgtatgtacagaatgtgaaatacagtagacaacaggttctccggacggtgacaacactccagtcatcagggcctgcccccatctgagcttcttcgcgcctggcaattgcagcacaagcgccaaccgcccttgttctcgtgttgcaccactacgtctcctttacagttccttgcaatatccatgtagcctgcccaccttcaacgtgccaaggccctcgcgcgaacttctcccgacctcgctcgctttgcgctttgctacttcttgtccccggtaccttgaaatttatcacaacattgtacattaagttactcgtagccagccaacatgccgcccccgggcaacgcgccgactgcaccagccgtgccgggcgcgttgcccgcctccacagagcaagtgttgaacgggctcgccgaggcgccgctggtggttcccccgcagctggtgcagtactacatgcgcaagagcggtcaggggccaatcctgtacaacatgagcgcggacgaccgggaggcaaatgaggacatgcgtttgtgagtgtgtgcaggggtggcggcacctgcagcacggttcccagcggatctcacctcgtcatgagcgccggcattcgccaccagttgccagtccttgaagcgcgtctgttaccgccatgcacgtgccgttcgctcacaggacccaggtcgtgagccttgcatcccagcgcttcctggccactgtgctcaatgacgccatgcagtgagtcccttgcctggacatgccgctcggcaccgcaccagccatgatttgcgggtcacggtatgatgtggatgcgctttgtgctgggtgtggagtcgctcagtgaggtccggggccgccgggtggagcggcccggagtacatagggcacggagaaggaagcctgcggcggttgctggcacgcccccaagtctatcgctagttgtatgtaaacaaatggtttgcaaatgcgggcggctgcacggagcacgcccctgttaccgcacacggcagcgcacggcatggcgctttggggcctgagccttccgcccaacacggctatacacacacacacacacacacacacacacacatgcatctgatgcatgttcctgtgtgtgataaggagcacaagtaaagcattacgcgtgcatgttctgtgtgtgtgtgtgtgtgtctggtaggtaccacaagatgaagcggggcgcgggtcccaaggccatgaaggaggcggggctggaccccaaggacaagcgccgcgtgctgcggactgaagacctggcggcggcgctgcagcaggaggtgcgcgccggagggctacagggtggtgcgcatgggtagagcgccggggaggggtgcgtaatgagtggatggttgggctgcggcggcggcggctctgcgccgcgctggtccagccgatgcatacacacgcacaacggcacaagtgttgtgcccggcacggtggcttgtgcgcgcggggtgcttctctgcacgcttcataacgctgcaccgcctgctggccggcgtcggtgtgacaatgcccctgctgcgtgcccctgctcgccggcgcagtacggtgtaaacatccgcaacccgccctactatgtggatgcgcgggacaaggaccaggcagcggccgggaggcggtagctggtggccacggcccatgtgaggggcacatgcatggactgacagcctcggtggtgcgcgcgtcgcggtgctggaaggggctgagcggcgcactaggggctagctgccattggtacgggcgtgtgcgtccgtcggtatggacgtgtgaagtgtccgggttgaaggcacatgcatcagggattggtaagcagctagcgtcatggatggacagcttcattgtggatgagtgcggtgaactcggcagcacgaagagacgaggcgtagatgatgctgcgtgcaggtgaggttgcgtgccttgggtgtcaggcatttggccccttgttcgcgaagtgcatggccttccgcggggtgagggggggagatatagcttactatgatgttgataatgatgtggcccctgctagctaacagaggtgagtaaaagtgcagagggcacgattgcatgggcttttgactatttaggctgactgacacgaccaatcacttatcgtaactgctgtcaaccaacgcgtctaggtatttcgtgcaccttgcgctggcacattatgcagtaccgttgaagcaattaaaagcaagcacccgctagctggcgttacagctgtgtcgttgcactgctgcatgctgttcaaatgactccaaaatagcgggatggaagggctgcgcacggcaggagaaggagggtgggctcatgcttccccttacaatttcagtccatcc

SEQ ID NO: 5 - DNA, 1176 bp, Chlamydomonas reinhardtii
tgttctcgtgttgcaccactacgtctcctttacagttccttgcaatatccatgtagcctgcccaccttcaacgtgccaaggccctcgcgcgaacttctcccgacctcgctcgctttgcgctttgctacttcttgtccccggtaccttgaaatttatcacaacattgtacattaagttactcgtagccagccaacatgccgcccccgggcaacgcgccgactgcaccagccgtgccgggcgcgttgcccgcctccacagagcaagtgttgaacgggctcgccgaggcgccgctggtggttcccccgcagctggtgcagtactacatgcgcaagagcggtcaggggccaatcctgtacaacatgagcgcggacgaccgggaggcaaatgaggacatgcgtttgacccaggtcgtgagccttgcatcccagcgcttcctggccactgtgctcaatgacgccatgcagtaccacaagatgaagcggggcgcgggtcccaaggccatgaaggaggcggggctggaccccaaggacaagcgccgcgtgctgcggactgaagacctggcggcggcgctgcagcaggagtacggtgtaaacatccgcaacccgccctactatgtggatgcgcgggacaaggaccaggcagcggccgggaggcggtagctggtggccacggcccatgtgaggggcacatgcatggactgacagcctcggtggtgcgcgcgtcgcggtgctggaaggggctgagcggcgcactaggggctagctgccattggtacgggcgtgtgcgtccgtcggtatggacgtgtgaagtgtccgggttgaaggcacatgcatcagggattggtaagcagctagcgtcatggatggacagcttcattgtggatgagtgcggtgaactcggcagcacgaagagacgaggcgtagatgatgctgcgtgcaggtgaggttgcgtgccttgggtgtcaggcatttggccccttgttcgcgaagtgcatggccttccgcggggtgagggggggagatatagcttactatgatgttgataatgatgtggcccctgctagctaacagaggtgagtaaaagtgcagagggcacgattgcatgggcttttgactatttaggctgactgacacgaccaatcacttatcgtaactgctgtcaaccaacgcgtctagg

SEQ ID NO: 6 - protein, 153 aa, Chlamydomonas reinhardtii
MPPPGNAPTAPVPGALPASTEQVLNGLAEAPLVVPPQLVQYYMRKSGQGPILYNMSADDREANEDMRLTQVVSLASQRFLATVLNDAMQYHKMKRGAGPKAMKEAGLDPKDKRRVLRTEDLAAALQQEYGVNIRNPPYYVDARDKDQAAAGRR

SEQ ID NO: 7 - DNA, 4198 bp, Chlamydomonas reinhardtii
  misc_feature (2282)..(2331): n = any nucleotide
atcatggcctgggtaatatttgcagtaataggcacaatcacttatgtgtctgtaccaggctgagcccaggaagtgcagtcttgcatgggttcgcacggttcgcaactgtgggtccgcgtcgacgatcgaacctcgaatcgtccgctatgaagtccatccttcatcgggcagtcgttttacaggctgaataccctcagcagctgtaaatcatttgcaccagcatacaccaaaatctattcgccttgaaaccaacggaccccttcgatctctctctggccactccaagctttggtcgttctgttttctgaccttgagaagcgctgccctctctacattgagctagtgtaagggccattgaacgactgcattttcctgcaagccatataccgctaggacgcccagtcgcagccgctggagcaatgacggagaccgaccaccgccgaagccgtccggactggtctcgcgcacagtcccttcgtctaattcagctccacgtcaagctgggtaacaggtgcgcgcgattgggctccgaattggaaaactaacaaaccaagccagttcgtgtgcgcgactccccgaagacaacagacccgctcaacctgcgctgctgtcctcgcaaattgattgcagttggaccgagatcgctaagcagctgcccggccgcactcgtgagtctgttttcagcgcagtcggggcgtcatgtcgcgacatgttgacgacggttggaggactttcagaagcgcgcgaggggcgagtacgcgactgacgtatgctgcaaagttatatgctttctcatatgcacggaactaccaacaaccttcgagcgtctcctgctctgtagtacgtctcagctcaccaccttctgtccccatgccattcgtatgcccccactacgtgcagagaatgactgcaagaatttcttcttcgggtgggtgccgttcggatgcatgtgcacgtacggtgcataccagggctagcctcgccttcaatcaaaccgcaccgaggtgctgaaccttcctccatcacacccctccctgcctatcggccacagagccctgcgcgcaaagcgcggctaccgtgacaacctggtctacgcctacgcgcgcgcgttgccgcccgcctccgcctctgcttgcgggtcgtgggagcaggacaagcgcggccccgacgccctcacccgtgccgccgcctacaaggcagccatgcaacaagtggcggcgcaagaagtggccgagcagatggagaagcagcagcgtagccagcagcaagagggagaggacggcggctgcggctcgggtgccgctggtgctactgccgaggacggcggggagccgggtgctgtagccgctgccagccgccgcagtagcagtgtgtcagtgggcgctgacggcgcggcgcccacggctcagggcgacggcatggacacgcaagaggacgccgcgtccgcgcctgcctgccccgcctcggctgccgcgagcccggttggtcctggtgagcagatggtagcacgagcgcgcgcctgtccgccccacgagcgcacacttgttgatggagttgttgctgcggatgccgttcggcctgcagttgttgccgtggtggtatgcgtgggtatggcgtgctcgccccacacatgaccgctaattgcatgctcatgcgtgtgcccgccgtgcttgcgcccggccaccaccagcaggtgacgtcagcgtccgccggctctcatccactggtgataccgtcgtcactgatgccgccggcaccaggactgttgttgccgctggtgttgttgctggcggttggcgctccgttgccgccgcggcgtcaatgccggcccaccctgccgccgtggtgtcgatgccgccggtggtgcccgcctctgttgtggcggcggccagcggcgtgcttggcgccgccgcggtgcccgctgctggtgcccctggtgaccggctgtccctgcagtcgctgcagccgccgccgcacggcttcgccgcccttccgcagtcggcggcgccggcgatcggcagcagcagcgccagtcccttctggcagcaccagcagcagcaccacctcatgggcccccgggtgcagcttctgtctcacgagtcgctggccctcctgcaccagcagcaccagcaggcgcagcagcactcgcacgtggtcctgcacgtggcgccgccgttcctgcagcagcaccaccagaacccgcaccaccagcacctgatggtgcagctggaaggcgccggcgccggtgcacctgccggcgccttccagctgcaacaccaccnnnnnnnnnnnnnnnnnnnnnnnnnnnnnnnnnnnnnnnnnnnnnnnnnnctccgcagcatgtgctgctgcctatggccgtccgcccgccgcacctgcttcagtacggcggtgcacacggtgccagtgccgctgcatctgctgccgccgctgctccgtctgcgggcatgggcgccttcgtcttccaccctcacccgcagcagcagcagctgccgcctgctgccgccgctgcctttgctgccgcctccgccgcgccgtcgcagcccgccgcagttgcggccgccgtgcactcgctggcacccgccgcctccgcagccctgtccctcagcggcagctcggtcctggaggcgaccaccaccaccacccgcatcacaaccaccactgccgcggctgttgcggccgctgctgctggcgccgcagtggctgctggggtcaagaccgagcccgcctcagccgaggcggccactggctgggcccagcagcagcaacagaaggcgcatgctggcgttagccgcagctgcagtagcagcagcagcagcagcgccgcctgcggcgcctgcagcacatgcaccgccggtgtcggcgctactcctgccacagcaacccagctgccgcaacaccagcaggatcaccagcttctgggcgacgactggtgcgccggtgacgaggagtgggccgagctcgggcgcatcctgcttggctgaagcagtgctgatgatggtggcaccttgcgaaggttgcatgaacgaatgacacaaacgcatactagtgtactgtagcctaaatggccggcctgaattctttgaagacaataagtaatttttgagcgtgagcgttctatggtacctagggcgccaccatgcaaactccacatgagtgagtggtatattacaaggcctggtccggacactgtgaaaccggttgacctaccgaacctaaatgtgagttgctcaaattcggagtagcttgaggtaggcgcatgaatgcatatggttcctgcagctgacttgctgcagcgtgggatgggatggcaaacgagttgtgtagccagcagcccaagggtgtctggatgtgttcaacctcctaatcgcatgcatgcgtgttggagtttgcaagcatgctcgcatgccgcactagccgcacacatacacaagaagactggagcaggcgagaattgcactgacacagagccaagcaagcccataaaaagtggtagctgtataagatgaggtgtacagtggatgggcagtgcccaaagcaagcaggggcgacagtgcaagccgcgggcactgccgtacagagtgctgtgtactgctgagatgctgcaaacggcagaagtagagcagccgggaaggctgtgggcggggaagagcgaacgtcggcatgtgtgagcgtcggtgtttacctagtgcggatgtgatgacgagcgagagagttgaccacagcggacatgcacggccttcggccccaccccatttttagggctggagttttcccaccatctctggagttgactcgcgaatgtgtcaaccacccgaaagaatggtcgcatgcttgtgagagttgctggaagactgctgagagcgagaccggatcccgcgtcaggggtgaaggtgcaaacggcacgaatgaacgcacttcatccaaggctcggaacagcacgcacgcatgtgcagttgtaaggcgctgcagcgatacgatgtaacttccttctcatgcagtcgtgtcacattgggctcagagcagccttgaaagacgcagtggtgcgggcagcaggtgcgcctggggcctctctggctgcccacggactgtaaatgtacggcgcctctctaccggaaagccccccctccacgacatggctcttggcccccaacggcccaacctactgcttgtaccgcggtcccaacactcatgtccg

SEQ ID NO: 8 - DNA, 3072 bp, Chlamydomonas reinhardtii
ctgtaaatcatttgcaccagcatacaccaaaatctattcgccttgaaaccaacggaccccttcgatctctctctggccactccaagctttggtcgttctgttttctgaccttgagaagcgctgccctctctacattgagctagtgtaagggccattgaacgactgcattttcctgcaagccatataccgctaggacgcccagtcgcagccgctggagcaatgacggagaccgaccaccgccgaagccgtccggactggtctcgcgcacagtcccttcgtctaattcagctccacgtcaagctgggtaacagttggaccgagatcgctaagcagctgcccggccgcactcagaatgactgcaagaatttcttcttcggagccctgcgcgcaaagcgcggctaccgtgacaacctggtctacgcctacgcgcgcgcgttgccgcccgcctccgcctctgcttgcgggtcgtgggagcaggacaagcgcggccccgacgccctcacccgtgccgccgcctacaaggcagccatgcaacaagtggcggcgcaagaagtggccgagcagatggagaagcagcagcgtagccagcagcaagagggagaggacggcggctgcggctcgggtgccgctggtgctactgccgaggacggcggggagccgggtgctgtagccgctgccagccgccgcagtagcagtgtgtcagtgggcgctgacggcgcggcgcccacggctcagggcgacggcatggacacgcaagaggacgccgcgtccgcgcctgcctgccccgcctcggctgccgcgagcccggttggtcctggtgacgtcagcgtccgccggctctcatccactggtgataccgtcgtcactgatgccgccggcaccaggactgttgttgccgctggtgttgttgctggcggttggcgctccgttgccgccgcggcgtcaatgccggcccaccctgccgccgtggtgtcgatgccgccggtggtgcccgcctctgttgtggcggcggccagcggcgtgcttggcgccgccgcggtgcccgctgctggtgcccctggtgaccggctgtccctgcagtcgctgcagccgccgccgcacggcttcgccgcccttccgcagtcggcggcgccggcgatcggcagcagcagcgccagtcccttctggcagcaccagcagcagcaccacctcatgggcccccgggtgcagcttctgtctcacgagtcgctggccctcctgcaccagcagcaccagcaggcgcagcagcactcgcacgtggtcctgcacgtggcgccgccgttcctgcagcagcaccaccagaacccgcaccaccagcacctgatggtgcagctggaaggcgccggcgccggtgcacctgccggcgccttccagctgcaacaccaccctccgcagcatgtgctgctgcctatggccgtccgcccgccgcacctgcttcagtacggcggtgcacacggtgccagtgccgctgcatctgctgccgccgctgctccgtctgcgggcatgggcgccttcgtcttccaccctcacccgcagcagcagcagctgccgcctgctgccgccgctgcctttgctgccgcctccgccgcgccgtcgcagcccgccgcagttgcggccgccgtgcactcgctggcacccgccgcctccgcagccctgtccctcagcggcagctcggtcctggaggcgaccaccaccaccacccgcatcacaaccaccactgccgcggctgttgcggccgctgctgctggcgccgcagtggctgctggggtcaagaccgagcccgcctcagccgaggcggccactggctgggcccagcagcagcaacagaaggcgcatgctggcgttagccgcagctgcagtagcagcagcagcagcagcgccgcctgcggcgcctgcagcacatgcaccgccggtgtcggcgctactcctgccacagcaacccagctgccgcaacaccagcaggatcaccagcttctgggcgacgactggtgcgccggtgacgaggagtgggccgagctcgggcgcatcctgcttggctgaagcagtgctgatgatggtggcaccttgcgaaggttgcatgaacgaatgacacaaacgcatactagtgtactgtagcctaaatggccggcctgaattctttgaagacaataagtaatttttgagcgtgagcgttctatggtacctagggcgccaccatgcaaactccacatgagtgagtggtatattacaaggcctggtccggacactgtgaaaccggttgacctaccgaacctaaatgtgagttgctcaaattcggagtagcttgaggtaggcgcatgaatgcatatggttcctgcagctgacttgctgcagcgtgggatgggatggcaaacgagttgtgtagccagcagcccaagggtgtctggatgtgttcaacctcctaatcgcatgcatgcgtgttggagtttgcaagcatgctcgcatgccgcactagccgcacacatacacaagaagactggagcaggcgagaattgcactgacacagagccaagcaagcccataaaaagtggtagctgtataagatgaggtgtacagtggatgggcagtgcccaaagcaagcaggggcgacagtgcaagccgcgggcactgccgtacagagtgctgtgtactgctgagatgctgcaaacggcagaagtagagcagccgggaaggctgtgggcggggaagagcgaacgtcggcatgtgtgagcgtcggtgtttacctagtgcggatgtgatgacgagcgagagagttgaccacagcggacatgcacggccttcggccccaccccatttttagggctggagttttcccaccatctctggagttgactcgcgaatgtgtcaaccacccgaaagaatggtcgcatgcttgtgagagttgctggaagactgctgagagcgagaccggatcccgcgtcaggggtgaaggtgcaaacggcacgaatgaacgcacttcatccaaggctcggaacagcacgcacgcatgtgcagttgtaaggcgctgcagcgatacgatgtaacttccttctcatg

SEQ ID NO: 9 - protein, 612 aa, Chlamydomonas reinhardtii
MTETDHRRSRPDWSRAQSLRLIQLHVKLGNSWTEIAKQLPGRTQNDCKNFFFGALRAKRGYRDNLVYAYARALPPASASACGSWEQDKRGPDALTRAAAYKAAMQQVAAQEVAEQMEKQQRSQQQEGEDGGCGSGAAGATAEDGGEPGAVAAASRRSSSVSVGADGAAPTAQGDGMDTQEDAASAPACPASAAASPVGPGDVSVRRLSSTGDTVVTDAAGTRTVVAAGVVAGGWRSVAAAASMPAHPAAVVSMPPVVPASVVAAASGVLGAAAVPAAGAPGDRLSLQSLQPPPHGFAALPQSAAPAIGSSSASPFWQHQQQHHLMGPRVQLLSHESLALLHQQHQQAQQHSHVVLHVAPPFLQQHHQNPHHQHLMVQLEGAGAGAPAGAFQLQHHPPQHVLLPMAVRPPHLLQYGGAHGASAAASAAAAAPSAGMGAFVFHPHPQQQQLPPAAAAAFAAASAAPSQPAAVAAAVHSLAPAASAALSLSGSSVLEATTTTTRITTTTAAAVAAAAAGAAVAAGVKTEPASAEAATGWAQQQQQKAHAGVSRSCSSSSSSSAACGACSTCTAGVGATPATATQLPQHQQDHQLLGDDWCAGDEEWAELGRILLG

SEQ ID NO: 10 - DNA, 5074 bp, Chlamydomonas reinhardtii
  misc_feature (2282)..(2331): n = any nucleotide
cgtacgtgtcagtccagtatgacggggggatgcgacggagtggatggggaacccccctgtctccagtgcaccctcagtgccgcttgtccagccatctttgcaaccccccatttccttgcaacccccaccccaccccccaggtgtttgagcagggcctgagccgcgtgcgcgcctgcctgtccaacgtggacagctcctgctgcctcatctgcctcaaccacatcgcgcccaccgaggcggtgtggcactgcggccgcggctgccacacagtgctgcacctggtgtgcatacaggtggggggcagggggcgggggagggggcagcagggagggggattaggctggggaatgagccgagcttgtgcagtccttgggcccacatcgcacaccataccaaccggtcgccgccgccgtgttgccgccatgttgccgccgtggcgccgcaggagtgggcgcgtagtcaggtggacgcggccaaggccaaggcggcggcgcggctgagtcagttccccgccgccggcgacgccgccgccgccgcggccgagtggggctgccccaagtgccgcgttacctaccccgcggccggcatacccagcacctacacatgcttttgcggcaaaggtgcgcgctctggggggggggggcgtctagcagaggtggggggctgggaaggtgggggctagggtcctggggggctggggtcctggggtttgggcggggtcctgtgtggcgcgtatgtgtcgtgcacgccttggggtttgcagcagcgctgtgctggccacgtgcccacgtgttccacacctttgtgtgtgtctgtgtgcgtgtgcttttatgtgtatgtgagtgtgtatgtgtgtgtgtgtttcctgacacgtgcgccgcccctcccctgcgcctctctctatcccacctctcccctccgcagccaccaaccccgagttcgacccatgggtggccccgcactcgtgcggcgaggtgtgcggccggcccctgcccggcggctgcggccacacctgcctgctgctgtgccaccccgggccctgcccgccctgcccgctggtggtggacgcgggctgctactgcggcgctcggcggctgaagcggcggtgcgggcaccacgagttcagctgcgagggtgtgtgcggagccaagctggagtgcgggcacaggtgggggcggggtggcagaggatgggggaggagggtggagggcgggggagtgggtggagggatcagggggtttcttgccaactgaagaggggtgcgggcgaggggcaaggatggctacggaagctcatgtgggggcggggctcgggcgcattcccttcgtgcacacggtgtcctgcgacagaggcgcagcgtactgcgtgtgtcaggccgcccccccttccgacctcaaccgccctgcccttgggctgagccgcgggagtccccaagtgccctagcttttccctccctcgcaaacttgtagcttcaataaacatcgccatctctgctcaccaacgtagcacatcaaaacaatcctgttcccgccaggtgtccggacatctgccacccagacgactgcgccccctgcgccgtgcccggcgacttccgctgccgctgcggcgccgaggcccggcggcgcggctgcggcgagcgcgattggcagtgcggccgcgtgtgcggcaaggcgctgggctgcggcagccacgtgtgtgagcgggtgtgccacgccggcccctgcggcgagtgccccttcgcgggcgtgcgcacctgcccctgcggaaaggtggagcacgtgggcatgggtgagtggccggcggggagggggagttgtagataccagcccaaactaaaccgaacccagtgcaaaagagcgaggaggagagcgaggaggggaagggggaagcgtgtgtatgtgtatgtgcgcgtgtgttgagaaagcacagggagaaagcaggggacagagtgtgtgcgtgtgcgcttatgtgctactgctgctgctgttgcttaagccccacgctgtggcctgcaatgcacctgcacctcccaatacacacatacacacacacacacaaacacacacacacacacacacacacaaacacacacacacacacacacacacacacacgcccgcccgcctgcacacaggctgcacggacaaggtgcccagctgcggtgccacgtgcggcaagctgctgccctgcggcgtgcacacctgcgccgaccgctgccaccagggcgagtgcagcgcgcagtgccgtgggcccgccgtgaagagctgccgctgcggcaagagccagaaggaggtgctgtgcttccaggtgggagactggatgtgttttgggggggagttgcattcatgagcaaaaagaagtgaaggtgcagccaggaacggtagtatagcagacgcgttgttgccgatgtccgtgaagtccagcagtgggggggcggtgagtggggttgggtggagtgggtgtgggtgtcggaggtgggtttgtcagccagggagcgcgtgcgtggcatatgcgtgtccgcgctatgtgatggtatagggcgcagttggttaacgttggaggtagagcaacctcggctgctagctcgccacactgagccaccggagctcttgcagtaacaaccaaaacgccgcccgcaggagttcacgtgcgagcggcgctgcaacgacatgcgcgcgtgcggccgccacccctgcaagcgccgctgctgcgacggcaactgcccgccgtgcgaggaggtgtgcggccgctggctcaagtgccgcaaccaccgctgccccgcgccctgccacagcgggccctgcaggtgtgcgcggatagcctacagctcgccagcggctgaggcgacatctcgcgggtttcatttgcggcacctaccagcccgacctactgctgctgcctgacccatcacgtgcacgtgaacaggctacaaaaacacaaacagacgcacacacacacaatctgccattcttttgcttcttgactttttgcttcttctgccccctccgcaggccctgcccgctgagcagcaccgtgacgtgtgcctgcggccgcgcctcctgcaccctgccctgcggcgccgaggacaaggccgagccgccgcactgcgccgcgccctgcgccgtgccgcgcctgtgccggcacgcgccctcgctgccgcgccaccgctgccacttcgggccctgcccgccctgcccgcagccctgcgccacgccgctggaatgcggccacgcctgctccgtgcccgggtgccacgacccgccgccgccgcccgtgcctgacttcgtgaagccggcggcgccgaaggcgccgtcagtggcggcggcggcggcggcggcggcggccgccgccgctggcagcgacggagccgtcagtgcgggtaacagtaagaagaaactgaatgcggcggcggcagtggaggtggcggcggcggcggcggtggcggcgccggccagcctggcggcgcagatgctggcggcggcggcggcgtccgggcagatgcccagcacctgcccgccctgctccgtgcggcagcaggtgcgtgcgtgtgtgttttgggtgtgagcgggggaggagggggagaggttggggaggagatttgctgcgcgcggtatggtatgcatctcggactcgggggcacaaaaggacgggtcttccgggtcgcagcagaagctgctgctgctgctgccgtacagtcacacacggccgctgcgatcaggaagattgggggaacagatgcatgggcgcgccgtgcgaagctgcgctccgctttacccaaccagatcgatgttaccattcaccgtcaacacacgatgccgccgccaccgtttcaccatcaacacacgatgccgcctacaccctgtgcccacacacaggtggcctgcgtgggcggccacaccaggctcgacctgccctgcgcctccgcgcgccccttcgcctgcggcgccgcctgcggccgcccgctcagctgcggcaaccactcctgcgcgctgccctgccacgccgtcgcatacgacccaatcaccatcgcgcgcgccgccgccgccgccgcggccggcggcgccgccgccgccgacccctcgctcccgcgcccctgccgccagtgcgagtcgcgctgcgaccggccgcgtcccgccggctgcgcgcacgcctgcccgttggcgtgccaccgcggcccctgcccgcgctgcgaggctccgatacggcacggctgcatgtgcggcaaatccacgctggcggtggcctgcgcggagctgggcgcggcggcggcggcggtggcggccggcggctcggcggcggcgctgtcgtgcggcaagccatgccacaggcagctgccctactgcccgcacccttgcaagtgagtcagcagccttttgaatgcggtttgccacatttgtgggaaccgtcacaccaatgccaggtccctcgtgcctgcacggacacccacgttgccggctccgtctgccaccaccgcagcccccaccgctctccacacggctgaggacaaccgcatggtctctctcaccgtcacccaccctttctcacctgcaccttcctacccgcgcccactcctccccccttaattaatcatgaatgtgacccccctcgggctactacttcctactacccccactccccccctcccgtctaccacttaactaatcatgaatgcaacccccgccttaattaatcatgaatgtactccctcactcgccccctaccgcagggcgctgtgccacgccggcgcctgccccgacccctccgcctgcgccgccgaggtgtcggtgcgctgcgcctgccccgccaagcgccgcgccaagtggcggtgcagcgaggtgcaggcggcgctggtggcgtcgggccgcccgcggtgaggggggaagagggagggagatggggagagagaggtagaggggacgatggggagagaaggagagagggacagagggacagagggacagagggcgatcgagagagagaggggcgagaagagggagagcgcgctgacgtggagcgtgtgcatgcgtttgtgcttgcccccttgatgaacgccagcttctcagcacagcaccgc

SEQ ID NO: 11 - DNA, 2019 bp, Chlamydomonas reinhardtii
tgcctcatctgcctcaaccacatcgcgcccaccgaggcggtgtggcactgcggccgcggctgccacacagtgctgcacctggtgtgcatacagttccccgccgccggcgacgccgccgccgccgcggccgagtggggctgccccaagtgccgcgttacctaccccgcggccggcatacccagcacctacacatgcttttgcggcaaagccaccaaccccgagttcgacccatgggtggccccgcactcgtgcggcgaggtgtgcggccggcccctgcccggcggctgcggccacacctgcctgctgctgtgccaccccgggccctgcccgccctgcccgctggtggtggacgcgggctgctactgcggcgctcggcggctgaagcggcggtgcgggcaccacgagttcagctgcgagggtgtgtgcggagccaagctggagtgcgggcacaggtgtccggacatctgccacccagacgactgcgccccctgcgccgtgcccggcgacttccgctgccgctgcggcgccgaggcccggcggcgcggctgcggcgagcgcgattggcagtgcggccgcgtgtgcggcaaggcgctgggctgcggcagccacgtgtgtgagcgggtgtgccacgccggcccctgcggcgagtgccccttcgcgggcgtgcgcacctgcccctgcggaaaggtggagcacgtgggcatgggctgcacggacaaggtgcccagctgcggtgccacgtgcggcaagctgctgccctgcggcgtgcacacctgcgccgaccgctgccaccagggcgagtgcagcgcgcagtgccgtgggcccgccgtgaagagctgccgctgcggcaagagccagaaggaggtgctgtgcttccaggagttcacgtgcgagcggcgctgcaacgacatgcgcgcgtgcggccgccacccctgcaagcgccgctgctgcgacggcaactgcccgccgtgcgaggaggtgtgcggccgctggctcaagtgccgcaaccaccgctgccccgcgccctgccacagcgggccctgcaggccctgcccgctgagcagcaccgtgacgtgtgcctgcggccgcgcctcctgcaccctgccctgcggcgccgaggacaaggccgagccgccgcactgcgccgcgccctgcgccgtgccgcgcctgtgccggcacgcgccctcgctgccgcgccaccgctgccacttcgggccctgcccgccctgcccgcagccctgcgccacgccgctggaatgcggccacgcctgctccgtgcccgggtgccacgacccgccgccgccgcccgtgcctgacttcgtgaagccggcggcgccgaaggcgccgtcaatgcccagcacctgcccgccctgctccgtgcggcagcaggtggcctgcgtgggcggccacaccaggctcgacctgccctgcgcctccgcgcgccccttcgcctgcggcgccgcctgcggccgcccgctcagctgcggcaaccactcctgcgcgctgccctgccacgccgtcgcatacgacccaatcaccatcgcgcgcgccgccgccgccgccgcggccggcggcgccgccgccgccgacccctcgctcccgcgcccctgccgccagtgcgagtcgcgctgcgaccggccgcgtcccgccggctgcgcgcacgcctgcccgttggcgtgccaccgcggcccctgcccgcgctgcgaggctccgatacggcacggctgcatgtgcggcaaatccacgctggcggtggcctgcgcggagctgggcgcggcggcggcggcggtggcggccggcggctcggcggcggcgctgtcgtgcggcaagccatgccacaggcagctgccctactgcccgcacccttgcaaggcgctgtgccacgccggcgcctgccccgacccctccgcctgcgccgccgaggtgtcggtgcgctgcgcctgccccgccaagcgccgcgccaagtggcggtgcagcgaggtgcaggcggcgctggtggcgtcgggccgcccgcggtga

SEQ ID NO: 12 - protein, 672 aa, Chlamydomonas reinhardtii
CLICLNHIAPTEAVWHCGRGCHTVLHLVCIQFPAAGDAAAAAAEWGCPKCRVTYPAAGIPSTYTCFCGKATNPEFDPWVAPHSCGEVCGRPLPGGCGHTCLLLCHPGPCPPCPLVVDAGCYCGARRLKRRCGHHEFSCEGVCGAKLECGHRCPDICHPDDCAPCAVPGDFRCRCGAEARRRGCGERDWQCGRVCGKALGCGSHVCERVCHAGPCGECPFAGVRTCPCGKVEHVGMGCTDKVPSCGATCGKLLPCGVHTCADRCHQGECSAQCRGPAVKSCRCGKSQKEVLCFQEFTCERRCNDMRACGRHPCKRRCCDGNCPPCEEVCGRWLKCRNHRCPAPCHSGPCRPCPLSSTVTCACGRASCTLPCGAEDKAEPPHCAAPCAVPRLCRHAPSLPRHRCHFGPCPPCPQPCATPLECGHACSVPGCHDPPPPPVPDFVKPAAPKAPSMPSTCPPCSVRQQVACVGGHTRLDLPCASARPFACGAACGRPLSCGNHSCALPCHAVAYDPITIARAAAAAAAGGAAAADPSLPRPCRQCESRCDRPRPAGCAHACPLACHRGPCPRCEAPIRHGCMCGKSTLAVACAELGAAAAAVAAGGSAAALSCGKPCHRQLPYCPHPCKALCHAGACPDPSACAAEVSVRCACPAKRRAKWRCSEVQAALVASGRPR

SEQ ID NO: 13 - DNA, 2097 bp, Chlamydomonas reinhardtii
  misc_feature (2282)..(2331): n = any nucleotide
gcaaggctgcgcggcggagtttgcgggtgtcaccaatcggtagctgcgtaaggcagccatgtcgagagtatcgtgtggccatatgtttaaggaactgcagtcggtgcaacatcccaaactgggattcgggcgcaatcacgaatttccttcattcccacgcgctcaggcctttacagctttctgataaactgtaacttatcaaaagcttaatcctttcacaatttactgcgagggcctgtgtaagttcacgtagtgacgtagagttcaggacgagtcactgctgtgcctgttgagacctatcaagcaccggcctagaaagcttgaaagaaagattacaggacttagatgggctagcctgctgcctcctggccttctcagaccaaagagctgacgcaatgaacggaactcagccgcgcctgtctggtgccacggcgccccctcctggactcgtgcaagtaccacaacctttcacgagcatgtggcctcagtactatgccaccggcggctcggcgccagcagtaatcggtgctactgacacgcgtgcggaacaggcggagcgcgacgcacgtaagctgaagcggaagcaggccaaccgggagagcgccaagcgcagcaagctgaagcggcaacaggcagagcgcgccctgcacgaagaggcgcggcgggtggagagcgagcgcgatggcctgacgtcgcagtacacggcagcgcagcagcggttgatggcggcgcagtcggcacagatggagctgcgacggaagatacagaagtacgctgcagctgacccggggccttctggtggcgggactgggagcgtgacgggcggcgcctcggcggcacccggcagcattacagagggcggcgaggcggccggtgttgacactggccccaccgaatcgtaacaggactcgtaacagcgccatgggtaacgtgcacgtggccgacgcctacccacgcgcgctccgcgctgtgacagcctgtagcatatagaacttgcacaacatgcggcacccgtatagctgaatgcttgaactgtgaactggtacgtagggcaaagctgccctgggtccccataggaggaaagatagttttggccccttgggcatgttcattgcttgcgcgttttcgtgtttagcaagtattagctctgtttgagctctgcgtatgtcgaactttgctagatggttgcgaggacttgattgtcgcccactggcgcatcgagccccggggcaaggggcgccagtaccgcggttgtgagtgtgagtaccagcctcaaggaaggtaacttaatgttgtcaaggcagccataacttacatggtgtatgagccagtatgtattgttgcaaacacgtgcttgtgaaggttttggtggtacctgcatgaccattctgtggtcgtgggggtgaaatgctagctgcggcttgcggagaatgcggaagataacaaagtcagtactgagttataggcgtatggaacggtgaggccgtgtgggtcccaagtgtgcaaagatgacttggtgagttttcctcccttctacagacggttgcggttttgaggcggcattaatcccatgcatgtttccgcgctgtgcccatggaccatgggatatgctcgtagttttgaagatgcgtgttgtatgtgagtcttggtactgctcggcttcctaattagaggtctccgactgcattgtcgtcgccgcatacgtgtgtgactatcaaacggtatgcatgcgcctgcctgggcccgtacgtggcagcgtgaagggcaggcagatggtaattagctattcggacgagcctccggactcagttgagttggtgggcccacacggactccgacggtagctgtgtggatgggaggaggacaaggtgtcgccagattgtgttacacaaggcacacgtctctttactatgcccagccttgacactggtgttagcacaccattcagctcagaaacagccttcatcggccgccacacttacacgccaaggcacacgtgtcgcaccactcctctgtgccagtagacactgcatggtcactttaaaatccacggccttcagatcaaaaatatcagcagatgataacatgatttgaagg

SEQ ID NO: 14 - DNA, 1697 bp, Chlamydomonas reinhardtii
aaaagcttaatcctttcacaatttactgcgagggcctgtgtaagttcacgtagtgacgtagagttcaggacgagtcactgctgtgcctgttgagacctatcaagcaccggcctagaaagcttgaaagaaagattacaggacttagatgggctagcctgctgcctcctggccttctcagaccaaagagctgacgcaatgaacggaactcagccgcgcctgtctggtgccacggcgccccctcctggactcgtgcaagtaccacaacctttcacgagcatgtggcctcagtactatgccaccggcggctcggcgccagcagtaatcggtgctactgacacgcgtgcggaacaggcggagcgcgacgcacgtaagctgaagcggaagcaggccaaccgggagagcgccaagcgcagcaagctgaagcggcaacaggcagagcgcgccctgcacgaagaggcgcggcgggtggagagcgagcgcgatggcctgacgtcgcagtacacggcagcgcagcagcggttgatggcggcgcagtcggcacagatggagctgcgacggaagatacagaagtacgctgcagctgacccggggccttctggtggcgggactgggagcgtgacgggcggcgcctcggcggcacccggcagcattacagagggcggcgaggcggccggtgttgacactggccccaccgaatcgtaacaggactcgtaacagcgccatgggtaacgtgcacgtggccgacgcctacccacgcgcgctccgcgctgtgacagcctgtagcatatagaacttgcacaacatgcggcacccgtatagctgaatgcttgaactgtgaactggtacgtagggcaaagctgccctgggtccccataggaggaaagatagttttggccccttgggcatgttcattgcttgcgcgttttcgtgtttagcaagtattagctctgtttgagctctgcgtatgtcgaactttgctagatggttgcgaggacttgattgtcgcccactggcgcatcgagccccggggcaaggggcgccagtaccgcggttgtgagtgtgagtaccagcctcaaggaaggtaacttaatgttgtcaaggcagccataacttacatggtgtatgagccagtatgtattgttgcaaacacgtgcttgtgaaggttttggtggtacctgcatgaccattctgtggtcgtgggggtgaaatgctagctgcggcttgcggagaatgcggaagataacaaagtcagtactgagttataggcgtatggaacggtgaggccgtgtgggtcccaagtgtgcaaagatgacttggtgagttttcctcccttctacagacggttgcggttttgaggcggcattaatcccatgcatgtttccgcgctgtgcccatggaccatgggatatgctcgtagttttgaagatgcgtgttgtatgtgagtcttggtactgctcggcttcctaattagaggtctccgactgcattgtcgtcgccgcatacgtgtgtgactatcaaacggtatgcatgcgcctgcctgggcccgtacgtggcagcgtgaagggcaggcagatggtaattagctattcggacgagcctccggactcagttgagttggtgggcccacacggactccgacggtagctgtgtggatgggaggaggacaaggtgtcgccagattgtgttacacaaggcac

SEQ ID NO: 15 - protein, 167 aa, Chlamydomonas reinhardtii
MNGTQPRLSGATAPPPGLVQVPQPFTSMWPQYYATGGSAPAVIGATDTRAEQAERDARKLKRKQANRESAKRSKLKRQQAERALHEEARRVESERDGLTSQYTAAQQRLMAAQSAQMELRRKIQKYAADPGPSGGGTGSVTGGASAAPGSITEGGEAAGVDTGPTES

SEQ ID NO: 16 - DNA, 22 bp, Artificial sequence
  synthetic
taattacntggtcaaaggaaac